(19) United States
(12) Patent Application Publication
(10) Pub. No.: US 2022/0072047 A1
(43) Pub. Date: Mar. 10, 2022

(54) MAGNETIC IMMUNO-PARTICLE AND USE THEREOF

(71) Applicant: UNIST(ULSAN NATIONAL INSTITUTE OF SCIENCE AND TECHNOLOGY), Ulsan (KR)

(72) Inventors: Joo Hun Kang, Ulsan (KR); Seyong Kwon, Ulsan (KR)

(73) Assignee: UNIST(ULSAN NATIONAL INSTITUTE OF SCIENCE AND TECHNOLOGY), Ulsan (KR)

(21) Appl. No.: 17/012,614

(22) Filed: Sep. 4, 2020

(51) Int. Cl. A61K 35/18; A61K 47/69; A61K 47/52; A61P 31/04; A61P 31/12; A61P 39/00; A61K 47/02; G01N 33/543; A61M 1/16; A61M 1/36

(52) U.S. Cl. CPC A61K 35/18; A61K 47/6901; A61K 47/52; A61P 31/04; A61P 31/12; G01N 2800/26; A61K 47/02; G01N 33/5434; A61M 1/16; A61M 1/3618; A61P 39/00

(57) ABSTRACT

Provided are magnetic immunoparticles and use thereof, specifically, magnetic immunoparticles including a cell membrane capable of capturing a pathogenic material and magnetic particles attached to the cell membrane, a method of detecting pathogenic materials using the magnetic immunoparticles, and a method of diagnosing and treating an infectious disease using the magnetic immunoparticles. The magnetic immunoparticles according to an aspect may include cell membranes capable of capturing pathogenic materials, and thus may minimize side effects in vivo, and may detect various kinds of pathogenic materials due to characteristics of the cells from which the cell membranes are derived. Further, since the magnetic immunoparticles include magnetic particles, the magnetic immunoparticles may be easily separated by applying a magnetic field, and thus pathogenic materials may be more effectively detected and removed.

### CROSS-REFERENCE TO RELATED APPLICATION

This application is based on and claims priority under 35 U.S.C. § 119 to Korean Patent Application No. 10-2018-0024727, filed on Feb. 28, 2018, in the Korean Intellectual Property Office, the disclosure of which is incorporated by reference herein in its entirety.

### BACKGROUND

### 1. Field

The present disclosure relates to magnetic immunoparticles and use thereof.

### 2. Description of Related Art

Worldwide, pathogenic microorganisms contaminate water supplies and infect humans, thus causing many diseases. Methods that have been developed to detect microorganisms present in blood have disadvantages in that the methods require a long time to detect harmful samples or do not accurately identify microorganisms. Representative methods of detecting microorganisms are a cell culture method and a polymerase chain reaction (PCR) method, which is a gene detection method.

The cell culture method is a method of separating and identifying pathogenic viruses and non-pathogenic viruses, and is determined by whether a cytopathic effect (CPE) occurs. In general, it takes a long time of 1 week to 4 weeks for the CPE to occur. For this reason, the cell culture method is ineffective in determining whether harmful microorganisms are present and preparing precautionary measures.

The PCR method, which is one of the genetic diagnostic methods, is a method of amplifying a small amount of DNA or RNA, and has very excellent sensitivity, specificity, and speed, as compared with the cell culture method, and therefore, is able to overcome the disadvantages of the cell culture method. However, there is a disadvantage in that when trace amounts of harmful microorganisms are present in a sample or when a large amount thereof is lost during a nucleic acid extraction process, the result of detection by the PCR method may be determined as negative. In other words, even though the microorganisms are not detected by the PCR method, there is a possibility that microorganisms may exist. In addition, the PCR method has fundamental problems in that quantitative analysis is difficult and there is a high risk of false positives due to contamination.

Treatment of diseases caused by infection still mostly relies on antibiotic administration. However, antibiotic administration has fatal disadvantages due to side effects of blood cell reduction, hypersensitivity reaction, neurotoxicity, cardiac toxicity, nephrotoxicity, and hepatotoxicity. In recent years, the emergence of ‘super bacteria’ that have resistance to antibiotics has led to a very low success rate in the treatment of infectious diseases using antibiotic administration.

To solve these problems, there is a need for the development of a method of isolating, detecting, or treating pathogenic microorganisms, the method having high detection or treatment efficiency of microorganisms while having safety since side effects do not occur when administered to the body.

### SUMMARY

An aspect provides magnetic immunoparticles.

Another aspect provides a composition or kit for detecting a pathogenic material, the composition or kit including the magnetic immunoparticles, or a method of detecting a pathogenic material using the magnetic immunoparticles.

Still another aspect provides a composition or kit for diagnosing an infectious disease, the composition or kit including the magnetic immunoparticles, or a method of diagnosing an infectious disease using the magnetic immunoparticles.

Still another aspect provides a composition for treating an infectious disease, the composition including the magnetic immunoparticles, a kit or device for treating an infectious disease using the magnetic immunoparticles, or a method of treating an infectious disease using the magnetic immunoparticles.

Additional aspects will be set forth in part in the description which follows and, in part, will be apparent from the description, or may be learned by practice of the presented embodiments of the disclosure.

An aspect provides magnetic immunoparticles including a cell membrane capable of capturing a pathogenic material; and magnetic particles attached to the cell membrane, wherein the cell membrane is derived from one or more selected from the group consisting of immune cells, red blood cells, endothelial cells, and epithelial cells.

The magnetic immunoparticles are magnetic particles attached to a cell membrane capable of capturing a pathogenic material, and may be used to capture various kinds of microorganisms or viruses and to separate the microorganisms or viruses using a magnetic field. The technology proposed in the present disclosure is 1) to prepare magnetic immunoparticles, in which magnetic particles are attached to a cell membrane capable of capturing pathogenic materials, 2) to attach various pathogenic materials to the magnetic immunoparticles by bringing the magnetic immunoparticles into contact with the pathogenic materials, and 3) to separate the magnetic immunoparticles to which the pathogenic materials are attached by a magnetic field.

As used herein, the term “attached” may refer to a form of being located outside or inside a cell membrane bilayer. For example, it may refer to a form in which magnetic particles are directly bound to the outside or inside of the cell membrane bilayer, or a form in which magnetic particles are absorbed into the cell membrane to be captured (or invaginated or enclosed), but is not limited thereto.

The pathogenic material may be one or more selected from the group consisting of pathogenic bacteria, fungi, viruses, parasites, prions, and toxins, but may include any pathogenic material without limitation, as long as it may be captured by the cell membrane. The pathogenic material may cause an infectious disease, e.g., malaria, in the body. In addition, the pathogenic material may include cells infected with the pathogenic material, e.g., red blood cells infected with malaria larvae, etc.

In one embodiment, the pathogenic bacteria may be any kind of Gram-positive bacteria or Gram-negative bacteria. More specifically, the pathogenic bacteria may include one or more selected from the group consisting of Enterococcus spp, Citrobacter spp, Staphylococcus spp, Klebsiella spp, Pseudomonas spp, Acinetobacter spp. Salmonella spp, Streptococcus spp, Escherichia spp, Mycobacterium spp, Mycoplasma spp, VIbrio spp, ShIgella spp. Campylobacter spp. Chlamydia spp, and bacteria that have acquired antibiotic resistance, but are not limited thereto.

In one specific embodiment, the pathogenic fungi may include one or more selected from the group consisting of Candida spp, Aspergillus spp, Trichophyton spp, and Cladophialophora spp, but are not limited thereto.

In one specific embodiment, the pathogenic viruses may include one or more selected from the group consisting of Adenoviridae, Picomaviridae, Herpesviridae, Hepadnaviridae, Flaviviridae, Retroviridae, Orthomyxoviridae, Paramyxoviridae, Papovaviridae, Polyomavirus, Rhabdoviridae, Togaviridae, Human Coronavirus 229E (HCoV229E), Cytomegalovirus (CMV), Severe acute respiratory syndrome coronavirus (SARS-CoV-1), SARS-CoV-2. Ebola Virus, and Dengue virus, but are not limited thereto.

Further, in one specific embodiment, the parasite may be malaria larvae.

Further, in one specific embodiment, the prions may be infectious protein pathogens that cause infectious diseases, including scrapie, mad cow disease, Creutzfeldt-Jakob disease, etc.

Further, in one specific embodiment, the toxins may include any one having pathogenicity, such as causing a disease, and may include pathogenic materials derived from the pathogenic bacteria, fungi, or viruses. For example, the toxins may include lipopolysaccharide (LPS), Zika virus (ZIKV) protein, SARS-CoV-2 Spike protein, etc.

In the present disclosure, the term “capturing” may include binding of the pathogenic material to the surface or inside of the cell membrane, and may refer to the binding of the cell membrane and the pathogenic material.

The cell membrane capable of capturing the pathogenic materials may be derived from one or more selected from the group consisting of immune cells, red blood cells, endothelial cells, and epithelial cells, but is not limited thereto.

As used herein, the term “immune cells” may refer to any cell that performs specific recognition/binding, non-specific binding, or phagocytosis with respect to an immunogen (e.g., a foreign immunogen and/or an endogenous immunogen) in the immune system of an organism (e.g., mammals, birds, fish, reptiles, amphibians, crustaceans, insects, etc.). Specifically, the immune cells may include one or more selected from the group consisting of neutrophils, eosinophils, basophils, monocytes, lymphocytes, Kupffer cells, microglias, macrophages, dendritic cells, mast cells, B cells, T cells, natural killer cells (NK cells), immune cell-derived cell lines, immune cell-like cells, and stem cell-derived immune cells, but are not limited thereto.

The immune cell-derived cell lines may include cell lines derived from immune cells, including HL60, U937, ML1, and THP-1 cell lines, etc., but are not limited thereto. The stem cell-derived immune cells refer to immune cells differentiated from stem cells by techniques known in the art.

The immune cells may be differentiated immune cells.

As used herein, the term “differentiated immune cells” refers to immune cells differentiated from progenitor cells of immune cells by stimulation such as lipopolysaccharide (LPS), dimethyl sulfoxide (DMSO), phorbol 12-myristate 13-acetate (PMA), retinoic acid, etc. The differentiated immune cells may have improved ability to capture pathogenic materials.

The immune cells may be immune cell-like cells. Specifically, the immune cell-like cells are cells derived from immune cells, cells isolated from tumor tissues, etc., or cells derived therefrom. The immune cell-like cells may be cells having the morphology or characteristics of immune cells. For example, the immune cell-like cells may be neutrophil- or macrophage (M0, M1, or M2)-like cells. According to one specific embodiment, the immune cell-like cells are cells obtained by differentiating leukemia cell lines, i.e., one or more selected from the group consisting of HL60, U937, THP-1, and K562 cell lines, and may be neutrophil- or macrophage (M0, M1, or M2)-like cells, but are not limited thereto.

As used herein, the term “red blood cells” refers to blood cells having a red flat disc shape, which carry oxygen through blood vessels to the systemic tissues and remove carbon dioxide.

As used herein, the term “endothelial cells” refers to cells that form the endothelium of the body's vessels (blood vessels, lymphatic vessels, etc.). For example, the endothelial cells may be one or more selected from the group consisting of lymphatic vessels, hepatic vessels, pulmonary vessels, cardiovascular vessels, renal vessels, cerebrovascular vessels, reproductive endothelium, and endothelial cells of the testis, but are not limited thereto. In one specific embodiment, the endothelial cells may be human hepatic sinusoidal endothelial cells.

As used herein, the term “epithelial cells” refers to cells that cover the body surface, the body cavities, or the inner surface of ducts. For example, the epithelial cells may be one or more selected from the group consisting of epithelial cells of lungs, intestines (stomach, duodenum, small intestine, large intestine), oral cavity, tongue, alveoli, lymphatic vessels, serosal membranes (pericardium, pleura, peritoneum, etc.), renal collecting tubules, thyroid glands, blood vessels, liver, salivary glands, skin epidermis, esophagus, vagina, sweat glands, germinal epithelium, testicular epithelium, follicles, exocrine glands, ureters, and bladder, but are not limited thereto. In one specific embodiment, the epithelial cells may be human oral epithelial cells or human intestinal epithelial cells.

The cell membrane capable of capturing pathogenic materials may be derived from cells of one or more individuals selected from the group consisting of primates such as humans, monkeys, etc., rodents such as rats, mice, etc., artiodactyla such as horses, cattle, pigs, sheep, goats, etc., mammals such as equine, canines, felines, etc., birds, fish, reptiles, amphibians, crustaceans, and insects, but is not limited thereto. Since the cell membrane capable of capturing pathogenic materials does not cause an immune rejection when administered to the body, it is excellent in safety when administered in vivo.

As used herein, the term “cell membrane” refers to a cell membrane of a cell itself, or a cell membrane separated from a cell through common methods, e.g., sonication, use of osmotic pressure difference, extrusion, etc.

The cell membrane may express one or more selected from the group consisting of lectins, Toll like receptors (TLRs), pattern recognition receptors (PRRs), duster of differentiation (CD) molecules, neutrophil extracellular traps (NETs), glycophorins, and cytokine receptors, but is not limited thereto. The cell membrane may express one or more selected from the group consisting of lectins, TLRs, PRRs, CD molecules, NETs, glycophorins, and cytokine receptors, thereby capturing or absorbing (uptake, endocytosis) magnetic particles or pathogenic materials, or binding or attaching to magnetic particles or pathogenic materials.

As used herein, the term “magnetic particles” refers to particles that may response to a magnetic field, and may be easily absorbed into cells, or bound, attached, introduced, invaginated, enclosed, or captured outside or inside the cell membrane. Specifically, the magnetic particles may include one or more magnetic elements selected from the group consisting of iron (Fe), nickel (NI), cobalt (Co), manganese (Mn), bismuth (Bi), zinc (Zn), strontium (Sr), lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), ruthenium (Lu), copper (Cu), silver (Ag), gold (Au), cadmium (Cd), mercury (Hg), aluminum (AD, gallium (Ga), indium (In), thallium (TI), calcium (Ca), barium (Ba), radium (Ra), platinum (Pt), and lead (Pd), but are not limited thereto.

The magnetic element may be oxidized or surface-modified. Specifically, iron may be oxidized to be included in the form of iron oxide in the magnetic immunoparticles. The surface modification may include surface modification with metals, surface modification with functional groups such as a carboxyl group or an amine group, surface modification with proteins such as antibody, streptavidin, or avidin, surface modification with carbohydrates, surface modification with polymers, and surface modification with lipids, but is not limited thereto. The magnetic particles may be stabilized by the above modification.

The magnetic particles may be used after being prepared through a known method, or commercially available magnetic particles may be purchased and used.

The magnetic particles may be used as they are, or may be used in a state where the magnetic particles are dispersed or suspended in an appropriate solvent (e.g., a buffer (PBS, saline, Tris-buffered saline, etc.)), but are not limited thereto.

Since the magnetic particles have a small particle size, each particle may contain a single magnetic domain. Therefore, the magnetic particles may exhibit superparamagnetism which is a magnetic property observed only when an external magnetic field exists. When the magnetic immunoparticles are prepared using magnetic particles exhibiting superparamagnetism, the magnetic immunoparticles may be simply and easily separated by applying an external magnetic field. Since the separation by applying a magnetic field is not affected by the surrounding environment such as pH, temperature, ions, etc., stability and sensitivity are excellent.

The magnetic particles may be selected from all magnetic particles having a particle size capable of being attached, introduced, invaginated, or enclosed into the cell membrane capable of capturing the pathogenic materials and exhibiting magnetism. For example, the magnetic particles may be magnetic particles having an average particle size of about 1 nm to about 30000 nm, about 10 nm to about 30000 nm, about 50 nm to about 30000 nm, about 100 nm to about 30000 nm, about 200 nm to about 30000 nm, about 300 nm to about 30000 nm, about 400 nm to about 30000 nm, about 500 nm to about 30000 nm, about 1 nm to about 20000 nm, about 10 nm to about 20000 nm, about 50 nm to about 20000 nm, about 100 nm to about 20000 nm, about 200 nm to about 20000 nm, about 300 nm to about 20000 nm, about 400 nm to about 20000 nm, about 500 nm to about 20000 nm, about 1 nm to about 10000 nm, about 10 nm to about 10000 nm, about 50 nm to about 10000 nm, about 100 nm to about 10000 nm, about 200 nm to about 10000 nm, about 300 nm to about 10000 nm, about 400 nm to about 10000 nm, about 500 nm to about 10000 nm, about 1 nm to about 5000 nm, about 10 nm to about 5000 nm, about 50 nm to about 5000 nm, about 100 nm to about 5000 nm, about 200 nm to about 5000 nm, about 300 nm to about 5000 nm, about 400 nm to about 5000 nm, about 500 nm to about 5000 nm, 1 nm to about 1000 nm, about 10 nm to about 1000 nm, about 50 nm to about 1000 nm, about 100 nm to about 1000 nm, about 200 nm to about 1000 nm, about 300 nm to about 1000 nm, about 400 nm to about 1000 nm, about 500 nm to about 1000 nm, about 1 nm to about 500 nm, about 10 nm to about 500 nm, about 50 nm to about 500 nm, about 100 nm to about 500 nm, about 200 nm to about 500 nm, about 300 nm to about 500 nm, or about 400 nm to about 500 nm, but are not limited thereto.

The magnetic particles may be included in a solution. The magnetic particles may be attached to the cell membrane while being included in a solution. In one specific embodiment, the solution may include a medium or a buffer used to culture or differentiate cells, or a combination thereof, and may be the same as a medium of magnetic immunoparticles.

The magnetic immunoparticles may include an outer surface including the cell membrane and an inner core including the magnetic particles.

In the magnetic immunoparticles, the inner core may include 1 or more, for example, 1 or more, and 1000000 or less magnetic particles. Specifically, the inner core may include 1 or more and 100000 or less, 1 or more and 10000 or less, 1 or more and 1000 or less, 1 or more and 100 or less, or 1 or more and 10 or less magnetic particles. The number of magnetic particles included in the inner core may be appropriately formed according to the size of magnetic particles or the size of magnetic immunoparticles. When the inner core includes two or more magnetic particles, the effect of absorbing and detecting the pathogenic materials may be improved.

An average particle size of the magnetic immunoparticles may be about 1 nm to about 30000 nm, about 10 nm to about 30000 nm, about 50 nm to about 30000 nm, about 100 nm to about 30000 nm, about 200 nm to about 30000 nm, about 300 nm to about 30000 nm, about 400 nm to about 30000 nm, about 500 nm to about 30000 nm, about 1 nm to about 20000 nm, about 10 nm to about 20000 nm, about 50 nm to about 20000 nm, about 100 nm to about 20000 nm, about 200 nm to about 20000 nm, about 300 nm to about 20000 nm, about 400 nm to about 20000 nm, about 500 nm to about 20000 nm, about 1 nm to about 10000 nm, about 10 nm to about 10000 nm, about 50 nm to about 10000 nm, about 100 nm to about 10000 nm, about 200 nm to about 10000 nm, about 300 nm to about 10000 nm, about 400 nm to about 10000 nm, about 500 nm to about 10000 nm, about 1 nm to about 5000 nm, about 10 nm to about 5000 nm, about 50 nm to about 5000 nm, about 100 nm to about 5000 nm, about 200 nm to about 5000 nm, about 300 nm to about 5000 nm, about 400 nm to about 5000 nm, about 500 nm to about 5000 nm, 1 nm to about 1000 nm, about 10 nm to about 1000 nm, about 50 nm to about 1000 nm, about 100 nm to about 1000 nm, about 200 nm to about 1000 nm, about 300 nm to about 1000 nm, about 400 nm to about 1000 nm, about 500 nm to about 1000 nm, about 1 nm to about 500 nm, about 10 nm to about 500 nm, about 50 nm to about 500 nm, about 100 nm to about 500 nm, about 200 nm to about 500 nm, about 300 nm to about 500 nm, or about 400 nm to about 500 nm, but is not limited thereto.

A thickness of the outer surface may be about 2 nm to about 30 nm, but is not limited thereto.

An average particle size of the inner core may be about 1 nm to about 30000 nm, about 10 nm to about 30000 nm, about 50 nm to about 30000 nm, about 100 nm to about 30000 nm, about 200 nm to about 30000 nm, about 300 nm to about 30000 nm, about 400 nm to about 30000 nm, about 500 nm to about 30000 nm, about 1 nm to about 20000 nm, about 10 nm to about 20000 nm, about 50 nm to about 20000 nm, about 100 nm to about 20000 nm, about 200 nm to about 20000 nm, about 300 nm to about 20000 nm, about 400 nm to about 20000 nm, about 500 nm to about 20000 nm, about 1 nm to about 10000 nm, about 10 nm to about 10000 nm, about 50 nm to about 10000 nm, about 100 nm to about 10000 nm, about 200 nm to about 10000 nm, about 300 nm to about 10000 nm, about 400 nm to about 10000 nm, about 500 nm to about 10000 nm, about 1 nm to about 5000 nm, about 10 nm to about 5000 nm, about 50 nm to about 5000 nm, about 100 nm to about 5000 nm, about 200 nm to about 5000 nm, about 300 nm to about 5000 nm, about 400 nm to about 5000 nm, about 500 nm to about 5000 nm, 1 nm to about 1000 nm, about 10 nm to about 1000 nm, about 50 nm to about 1000 nm, about 100 nm to about 1000 nm, about 200 nm to about 1000 nm, about 300 nm to about 1000 nm, about 400 nm to about 1000 nm, about 500 nm to about 1000 nm, about 1 nm to about 500 nm, about 10 nm to about 500 nm, about 50 nm to about 500 nm, about 100 nm to about 500 nm, about 200 nm to about 500 nm, about 300 nm to about 500 nm, or about 400 nm to about 500 nm, but is not limited thereto.

In the magnetic immunoparticles, the cell membrane may form a vesicle. As used herein, the term “vesicle” may refer to particles formed by self-assembly or reassembly by extrusion of cell membrane which is separated by extracting (separating) the cell membrane from cells by a known technique such as sonication, osmotic pressure difference, or extrusion.

A schematic illustration of a process of preparing magnetic immunoparticles according to a specific embodiment is shown in FIG. 1. Specifically, according to Type 1 method of FIG. 1, cells extracted from animals are added to a solution containing magnetic particles (e.g., blood, aqueous solution, purified water, buffer, medium, etc.), and the magnetic particles are allowed to be absorbed (or included, integrated) inside the cells by a cellular uptake phenomenon, and as a result, cells containing magnetic particles therein (e.g., magnetic immune cells) or cell analogs may be generated. The cells containing magnetic particles therein (e.g., magnetic immune cells) may perform their original functions (e.g., functions of immune cells), and at the same time, may exhibit magnetism by magnetic particles contained therein or may be affected by a magnetic field.

In addition, according to Type 2 method of FIG. 1, magnetic immunoparticles may be generated using the cell membrane separated (or purified) from cells. Specifically, the cell membranes are separated (or purified) from cells through a common method (e.g., using the osmotic pressure difference, sonication, extrusion, etc.), and the cell-derived membrane obtained therefrom and magnetic particles are extruded, and the magnetic particles are included inside the cell-derived membrane, and as a result, magnetic immunoparticles including the magnetic particles inside the cell-derived membrane may be generated. More specifically, to separate (or purify) the cell membrane from the cells, the cells are added to a low osmotic pressure solution, and the low osmotic pressure solution moves into the cells to swell the cells while forming pores in the cell membrane, through which intracellular organelles escape into the extracellular environment. The intracellular organelles that have escaped through the pores are separately removed by centrifugation, and only the cell-derived membranes are separated (or purified), and the separated (or purified) cell-derived membranes may be sonicated to be split into a smaller size. The cell-derived membranes and the magnetic particles are mixed and subjected to an extrusion process to generate magnetic immunoparticles including the magnetic particles inside the cell-derived membranes. Since the generated magnetic immunoparticles include cell membrane components (lipid bilayer, membrane protein, etc.) of cells in the outer membrane, they may perform functions similar to the cells (e.g., functions of immune cells). In addition, the generated magnetic immunoparticles may exhibit magnetism or may be affected by a magnetic field, due to magnetic particles included therein.

In addition, according to Type 3 method as shown in FIG. 1, magnetic immunoparticles may be generated using the cell membrane separated (or purified) from cells. Specifically, the cell membranes are separated (or purified) through a common method (e.g., using the osmotic pressure difference, extrusion, etc.), and the cell-derived membranes obtained therefrom and magnetic particles are sonicated, and the magnetic particles are included inside the cell-derived membranes, and as a result, magnetic immunoparticles including the magnetic particles inside the cell-derived membranes may be generated. More specifically, to separate (or purify) the cell membrane from the cells, the cells are added to a low osmotic pressure solution, and the low osmotic pressure solution moves into the cells to swell the cells while forming pores in the cell membrane, through which intracellular organelles escape into the extracellular environment. The intracellular organelles that have escaped through the pores are separately removed by centrifugation, and only the cell-derived membranes are separated (or purified), and the separated (or purified) cell-derived membranes and the magnetic particles may be mixed and sonicated to generate magnetic immunoparticles Including the magnetic particles inside the cell-derived membranes. Since the generated magnetic immunoparticles include cell membrane components (lipid bilayer, membrane protein, etc.) of cells in the outer membrane, they may perform functions similar to the cells (e.g., functions of immune cells). In addition, the generated magnetic immunoparticles may exhibit magnetism or may be affected by a magnetic field, due to magnetic particles included therein.

Another aspect provides a composition for detecting pathogenic materials, the composition including the magnetic immunoparticles.

As used herein, the term “detecting pathogenic materials” may refer to including determining whether the pathogenic materials are present in a sample, or detecting the pathogenic materials present in the sample.

The magnetic immunoparticles may be used in detecting pathogenic materials by binding the pathogenic materials to cell membranes or capturing the pathogenic materials into the cel membranes by characteristics of the cells, from which the cell membranes are derived, using the cell membranes capable of capturing the pathogenic materials.

In the case of magnetic immunoparticles using existing targeting substances (e.g., antibodies), it is possible to bind to the pathogenic materials by forming a targeting substance capable of binding to the pathogenic materials on the surface or inside of the magnetic particles. Therefore, it is difficult to effectively target a pathogenic material without accurate information of a specific antigen for the pathogenic material to be targeted, and there is a great difficulty in targeting many kinds of pathogenic materials at the same time. In addition, since an antibody must be used, there is a problem in that the synthesis is very difficult and the cost is high.

In the magnetic immunoparticles, cells isolated from a living body, e.g., immune cells, cells having immune actions (cells participating in immune activity, immune cell-derived cells, immune cell-like cells, etc.) or cell membranes derived therefrom may be used as they are, and therefore, there is an advantage in that the system and characteristics of the cells themselves, e.g., the system and characteristics of immune cells or cells having immune actions, such as various opsonin protein systems produced by the immune system, may be used to detect various kinds of unknown pathogenic materials (microorganisms or viruses) at once. Accordingly, the magnetic immunoparticles may be used in detecting various pathogenic materials.

The composition may rapidly detect contaminants (bacteria, fungi, viruses, other microorganisms, toxins (e.g., endotoxins, etc.), or contaminant compounds) present in trace amounts in drinking water, various foods including beverages, hygiene products, environmental samples, etc., and therefore, the composition may be used in a safety test of foods, hygiene products, environmental samples, etc. In addition, the composition may detect pathogenic materials present in a living body.

The magnetic immunoparticles according to one specific embodiment may detect pathogenic materials present in diabetic blood. Therefore, the composition for detecting pathogenic materials, the composition including the magnetic immunoparticles, may be used in detecting pathogenic materials present in the blood of an individual with diabetes.

Still another aspect provides a composition for diagnosing an infectious disease, the composition including the magnetic immunoparticles. The infectious disease may be one or more selected from the group consisting of systemic or local infections, inflammation, sepsis, and poisoning by toxins, but is not limited thereto. In addition, any disease caused by infection with the above-described pathogenic materials is included without limitation. Specifically, the infectious disease may be one or more selected from the group consisting of malaria infection (Malaria Journal 2012, 11:343; Blood Adv. 2019, 3 (11): 1761-1773), Mycobacterium tuberculosis, pneumonia, food poisoning, tetanus, typhoid, diphtheria, syphilis, Hansen's disease, Chlamydia infection, smallpox, influenza, epidemic parotitis, measles, chickenpox, Ebola, rubella, Coronavirus infection, scrapie, mad cow disease, and Creutzfeldt-Jakob disease, but is not limited thereto.

As used herein, the term “diagnosis of infectious disease” may refer to determining whether an individual currently or previously has an infectious disease, or determining whether an individual has been infected with a pathogenic material that may cause an infectious disease.

The composition may be used in diagnosing an infectious disease of an individual by detecting pathogenic materials captured by the magnetic immunoparticles. In addition, the composition may be used in diagnosing an infectious disease of an individual with diabetes by detecting pathogenic materials captured by magnetic immunoparticles in diabetic blood.

Still another aspect provides a composition for removing pathogenic materials, the composition including the magnetic immunoparticles. The composition may be used in removing pathogenic materials from a sample by applying a magnetic field to the pathogenic material-captured magnetic immunoparticles and separating the pathogenic material-captured magnetic immunoparticles from the sample using the applied magnetic field.

In addition, the composition may be used in removing pathogenic materials in the blood of an individual with diabetes by removing the pathogenic material-captured magnetic immunoparticles from the diabetic blood.

Among the terms or elements mentioned in the composition, those the same as mentioned in the description of the magnetic immunoparticles are understood to be the same as mentioned in the above description of the magnetic immunoparticles.

Still another aspect provides a method of detecting a pathogenic material, the method including bringing the magnetic immunoparticles into contact with a sample and mixing the magnetic immunoparticles with the sample, and applying a magnetic field to the mixed sample.

Still another aspect provides a method of removing a pathogenic material, the method including bringing the magnetic immunoparticles into contact with a sample and mixing the magnetic immunoparticles with the sample, and applying a magnetic field to the mixed sample.

Still another aspect provides a method of diagnosing an infectious disease or providing information about diagnosis, the method including bringing the magnetic immunoparticles into contact with a sample and mixing the magnetic immunoparticles with the sample, and applying a magnetic field to the mixed sample.

In the method, the sample may be one or more selected from the group consisting of biological samples (e.g., body fluid such as blood (e.g., whole blood), plasma, serum, lymph, cerebrospinal fluid, etc., cells, or tissues) present in or separated from a living body of an animal (including or not including humans), drinking water (e.g., ground water, tap water, bottled water, purified water, mineral water, etc.), various foods, various hygiene products that directly act on the living body, tableware, kitchen supplies, environmental samples (e.g., soil, sea water, river water, etc.), but is not limited thereto. The sample may be all targets requiring detection, removal, and/or diagnosis of pathogenic materials. The sample itself may be a fluid or may be in the form of a suspension, in which the sample is suspended in an appropriate medium (e.g., purified water, sterile buffer, etc.).

When the sample is a biological sample separated from the living body, drinking water, various foods, various hygiene products that directly act on the living body, tableware, kitchen supplies, environmental samples, etc., the bringing of the magnetic immunoparticles into contact with the sample and the mixing of the magnetic immunoparticles with the sample may be performed in vitro. The method may include incubating, in vitro, the magnetic immunoparticles together with the sample.

When the sample is a body fluid such as blood, a cell, or a tissue present in a living body of an animal (including or not including humans), the bringing of the magnetic immunoparticles into contact with the sample and the mixing of the magnetic immunoparticles with the sample may be performed in vitro or in vivo. When the bringing of the magnetic immunoparticles into contact with the sample and the mixing of the magnetic immunoparticles with the sample are performed in vitro, it may include separating the sample present in the living body from the living body, and bringing the magnetic immunoparticles into contact with the separated sample and mixing the magnetic immunoparticles with the sample in vitro, and also, as described above, the bringing of the magnetic immunoparticles into contact with the sample and the mixing of the magnetic immunoparticles with the sample may include incubating, in vitro, the magnetic immunoparticles together with the separated sample.

When the bringing of the magnetic immunoparticles into contact with the sample and the mixing of the magnetic immunoparticles with the sample are performed in vivo, it may include administering (or injecting) the magnetic immunoparticles into a circulatory organ (e.g., blood vessels (blood), etc.) of a target individual (a vertebrate animal including or not including humans). In addition, even when the bringing of the magnetic immunoparticles into contact with the sample and the mixing of the magnetic immunoparticles with the sample are performed in vivo, it may further include bringing the magnetic immunoparticles into contact with the sample separated from the target individual and mixing the magnetic immunoparticles with the sample in vitro. In addition, as described above, it may further include incubating, in vitro, the magnetic immunoparticles together with the sample separated from the target individual.

The incubating in vitro may be performed under common conditions using a medium, a buffer solution, a saline solution, or drinking water, which is commonly used for cell culture, and the biological sample. For example, the incubating may be performed for 1 second to 96 hours, 1 second to 48 hours, 1 second to 24 hours, e.g., 1 second to 12 hours, 1 second to 6 hours, 1 second to 120 minutes, or 1 second to 60 minutes under the temperature condition of 0° C. to 40° C. or 2° C. to 38° C. The incubating may be performed using an appropriate medium, buffer solution, saline solution, or biological sample.

In addition, the method may include applying a magnetic field to the mixed sample, in addition to bringing the magnetic immunoparticles into contact with the sample and mixing the magnetic immunoparticles with the sample. The applying of the magnetic field may be performed in vitro. When the sample is a biological sample present in a living body (i.e., when the magnetic immunoparticles are administered to the living body (in the circulatory organ)), in order to apply the magnetic field, the biological sample (e.g., blood, etc.), to which the magnetic immunoparticles have been administered, may be separated (extracted) from the living body. In this case, the method may further include separating (or extracting) the biological sample, to which the magnetic immunoparticles have been administered, from the living body, before applying the magnetic field.

The method may further include separating (or extracting) the magnetic immunoparticles from the sample using the applied magnetic field (magnetic force), after applying the magnetic field to the mixed sample. At this time, the separated magnetic immunoparticles may be bound to pathogenic materials in the sample or may be in a state in which the pathogenic materials in the sample are captured therein. This may be performed in vitro, and through this, pathogenic materials may be removed from the sample.

The method may further include injecting back the sample, from which the magnetic immunoparticles have been removed, from the outside of the body into the body, after applying a magnetic field to the mixed sample or separating (or extracting) the magnetic immunoparticles from the sample using the applied magnetic field (magnetic force). In this regard, the sample that is injected back from the outside of the body into the body may be a sample, from which the pathogenic materials have been removed by binding the pathogenic materials to the magnetic immunoparticles or by capturing the pathogenic materials inside the magnetic immunoparticles.

The method may further include analyzing the pathogenic materials captured by magnetic immunoparticles separated (or removed) from the sample. The analysis may be performed through a means which is commonly used in analyzing the pathogenic materials (e.g., bacteria, fungi, viruses, parasites, prions, toxins (e.g., endotoxin), etc.).

In the above method, to facilitate measurement of the magnetic immunoparticles, the magnetic immunoparticles may be obtained by using cells (cell membranes) and/or magnetic particles labeled with a detectable labeling material. The labeling material may be any material (small molecular compounds or proteins or poly/oligo peptides, etc.) detectable by a common method, and may be, for example, one or more selected from the group consisting of fluorescent materials, light-emitting materials, etc.

The method may be performed in vitro by applying a magnetic immunoparticle-based hemodialysis or magnetic immunoparticle-based extracorporeal circulation method. Specifically, the magnetic immunoparticle-based hemodialysis or magnetic immunoparticle-based extracorporeal circulation method may be performed by a method shown in FIG. 4, 10, 16, or 18, or a kit or device using the same, but is not limited thereto.

Therefore, in the method, the bringing of the magnetic immunoparticles into contact with the sample and the mixing of the magnetic immunoparticles with the sample are to allow the pathogenic materials present in the sample to bind with the magnetic immunoparticles or to allow uptake of the pathogenic materials into the magnetic immunoparticles, and may include applying the magnetic immunoparticles and the sample to a reaction unit, and forming a complex of the magnetic immunoparticles and the pathogenic materials by capturing the pathogenic materials of the sample inside the magnetic immunoparticles in the reaction unit. The applying to the reaction unit and the forming the complex may be performed in vitro. According to one specific embodiment, the reaction unit may be a reaction unit included in the kit or device using the magnetic immunoparticle-based hemodialysis or magnetic immunoparticle-based extracorporeal circulation method shown in FIG. 4, 10, 16, or 18.

In addition, in the method, the applying of the magnetic field to the mixed sample is to capture the magnetic immunoparticles, which bound to the pathogenic materials or captured the pathogenic materials internally, and may include applying the reaction product of the magnetic immunoparticles and the sample to a magnetic field-forming unit including a substrate capable of forming the magnetic field, and forming the magnetic field on the substrate. In the above procedure, the magnetic field may be formed (applied) by any common method. For example, it may be performed using a magnet, such as an electromagnet by electromagnetic induction or a permanent magnet. One or more magnets may be included, and may be applied in various arrangements such as arrangement in series, parallel, or circular shape, etc. The applying to the magnetic field-forming unit and the forming the magnetic field may be performed in vitro. According to one specific embodiment, the magnetic field-forming unit may be a magnetic field-forming unit included in the kit or device using the magnetic immunoparticle-based hemodialysis or magnetic immunoparticle-based extracorporeal circulation method shown in FIG. 4, 10, 18, or 18.

Among the terms or elements mentioned in the method, those the same as mentioned in the description of the magnetic immunoparticles or the composition are understood to be the same as mentioned in the above description of the magnetic immunoparticles or the composition.

Still another aspect provides a kit for detecting pathogenic materials, the kit including the magnetic immunoparticles.

Still another aspect provides a kit for removing pathogenic materials, the kit including the magnetic immunoparticles.

Still another aspect provides a kit for diagnosing or treating an infectious disease, the kit including the magnetic immunoparticles.

The kit may further include a reaction unit; and a magnetic field-forming unit.

As used herein, the term “reaction unit” may refer to a unit where the magnetic immunoparticles and the sample are brought into contact with each other to be mixed, Incubated, or reacted, or a reaction product is injected, the reaction product obtained from a reaction by bringing the magnetic immunoparticles into contact with the sample. In the kit, the magnetic immunoparticles may be included in the reaction unit, or may be applied to the reaction unit in the form of a reactant obtained by previously reacting the magnetic immunoparticles with a sample, or may be provided separately from the reaction unit, wherein the magnetic immunoparticles may be provided in the form of a dispersion of being dispersed in an appropriate medium (e.g. buffer).

As used herein, the term “magnetic field-forming unit” may refer to a unit that forms a magnetic field. The magnetic field-forming unit may be included in the reaction unit, may be provided separately from the reaction unit, or may be integrated into the reaction unit in whole or in part. When the magnetic field-forming unit exists separately from the reaction unit, the reaction unit and the magnetic field-forming unit may be connected with each other by a channel through which a fluid may move. Magnetic particles attached to magnetic immunoparticles may move to the magnetic field-forming unit by the magnetic field which is formed by the magnetic field-forming unit, and thus the magnetic immunoparticles may be separated. In this regard, the separated magnetic immunoparticles may be magnetic immunoparticles in which pathogenic materials have been captured. For example, the magnetic field-forming unit may include one or more means for applying a magnetic field, such as a magnet (e.g., an electromagnet by electromagnetic induction, a permanent magnet, etc.), and the magnetic field-forming unit may exist separately from the reaction unit, or may be integrated into the reaction unit in whole or in part. The shape of the reaction unit and/or the magnetic field-forming unit which are integrated or provided separately from each other is not particularly limited, and may have various shapes, such as a well form, a plate form, a channel form, etc. The number of each of the reaction unit and/or the magnetic field-forming unit which are integrated or provided separately from each other is also not particularly limited, and may be one or more. For example, when the reaction unit and the magnetic field-forming unit are provided separately from each other, one or more, for example, 1 to 10 reaction units may be connected to 1 to 10 magnetic field-forming units, or when the reaction unit and the magnetic field-forming unit are integrated, 1 to 10 of the integral form of the reaction unit and the magnetic field-forming unit may be provided, but are not limited thereto. When the magnetic field-forming unit exists separately from the reaction unit, the reaction unit and the magnetic field-forming unit may be connected with each other by a channel through which a fluid may move.

The kit may include one or more injection units connected to the reaction unit, the injection unit into which a sample (in a fluid state, such as a body fluid, etc.) and/or the magnetic immunoparticles (e.g., in a dispersion state), or a reaction product thereof may be injected. The other side of the injection unit, which is not connected to the reaction unit, may be directly connected to an individual, or may be connected to a sample isolated from the individual, and thus, the sample may be injected through the injection unit. Further, the magnetic immunoparticles may be injected through the other side of the injection unit, to which the reaction unit is not connected.

The kit may further include one or more discharge units connected to the magnetic field-forming unit, the discharge unit for discharging the magnetic immunoparticles captured by a magnetic field. The discharge unit may further include a detection unit including a detection means capable of detecting the pathogenic materials captured by the discharged magnetic immunoparticles.

In the magnetic field-forming unit of the kit, the pathogenic material-captured magnetic immunoparticles may be separated from the sample by a magnetic field, and may be separately collected or concentrated. In this case, the pathogenic material-captured magnetic immunoparticles may not be discharged, and may be filtered, collected, concentrated, or removed in the magnetic field-forming unit or in a collecting unit connected to the magnetic field-forming unit.

The kit may further include a sample discharge unit.

As used herein, the term “sample discharge unit” may refer to a unit, from which the sample separated by applying a magnetic field thereto is discharged. The sample discharge unit may be one or more. The sample that has moved to one place to be separated by the magnetic field application in the presence of a fluid flow, for example, the sample from which the pathogenic material-captured magnetic immunoparticles have been removed may be discharged through the sample discharge unit, and may be separately concentrated or separated.

In the kit, the sample discharge unit may be connected to the injection unit or may be connected to an individual. Therefore, the pathogenic material-removed sample which has been discharged through the sample discharge unit is injected again through the injection unit, and the pathogenic materials in the sample are removed. This procedure is repeated to more effectively remove the pathogenic materials in the sample that have not been completely removed. In addition, the pathogenic material-removed sample which has been discharged through the sample discharge unit may be injected back into the individual. According to one specific embodiment, the sample discharge unit and the discharge unit may be separated and referred to as a first discharge unit and a second discharge unit.

A schematic illustration of the kit according to one specific embodiment is shown in FIG. 4, but is not limited thereto.

Among the terms or elements mentioned in the kit, those the same as mentioned in the description of the magnetic immunoparticles, the composition, or the method are understood to be the same as mentioned in the above description of the magnetic immunoparticles, the composition, or the method.

Still another aspect provides a composition for treating an infectious disease, the composition including the magnetic immunoparticles. The composition may be used in treating an infectious disease of an individual by detecting and removing pathogenic materials present in a living body by the magnetic immunoparticles included in the composition. In addition, the composition may be used in treating an infectious disease of an individual with diabetes by detecting and removing pathogenic materials captured by the magnetic immunoparticles in the diabetic blood. The composition for treating an infectious disease may be applied to magnetic immunoparticle-based hemodialysis or magnetic immunoparticle-based extracorporeal circulation.

Still another aspect provides a method of treating an infectious disease, the method including bringing the magnetic immunoparticles into contact with a sample separated from an individual and mixing the magnetic immunoparticles with the sample, and removing pathogenic materials by applying a magnetic field to the mixed sample. The method may further include injecting the sample, from which the pathogenic materials have been removed, back into the individual.

The method may be applied to magnetic immunoparticle-based hemodialysis or magnetic immunoparticle-based extracorporeal circulation method. According to one specific embodiment, a schematic illustration of the magnetic immunoparticle-based hemodialysis or magnetic immunoparticle-based extracorporeal circulation method is as shown in FIG. 4, 10, 16, or 18, but is not limited thereto, and it may be modified, as long as the same principle is applied.

The method of treating an infectious disease according to a specific embodiment may be implemented in the form of a fluid device. In addition, a technical configuration mentioned below may be provided as a single or multiple configurations depending on the type of application.

More specifically, as shown in FIGS. 18 and 19, a fluidic device 100 may include a first injection unit 110 to which a sample (e.g., blood) is supplied, a second injection unit 120 to which magnetic particle-bound cell membranes (hereinafter, referred to as “magnetic immunoparticles”) are supplied, a reaction unit 130 in which the first injection unit 110 and the second injection unit 120 are combined into one unit, and pathogenic materials in the sample are mixed with the magnetic immunoparticles, a magnetic field-forming unit 140 in which a material capable of applying a magnetic field is disposed at one side to induce to move the magnetic immunoparticles to one side of the magnetic field-forming unit 140, a first discharge unit (also referred to as a “sample discharge unit”) 150 through which the sample from which at least a part of the pathogenic materials has been removed is discharged, and a second discharge unit 160 through which the pathogenic material-captured magnetic immunoparticles are discharged.

The first injection unit 110 receives a sample (e.g., blood) from the outside, wherein the sample may include pathogenic materials. According to one specific embodiment, as shown in FIG. 18, the first injection unit 110 may be connected to a patient's body.

The second injection unit 120 forms an inlet separately from the first injection unit 110, through which magnetic particle-bound cell membranes (magnetic immunoparticles) may be supplied.

The reaction unit 130 may be formed by combining the branched first injection unit 110 and second injection unit 120 into one unit. Accordingly, the pathogenic materials in the sample supplied through the first injection unit 110 may be mixed with the magnetic particle-bound cell membranes (magnetic immunoparticles) supplied through the second injection unit 120. Further, in the reaction unit 130, the pathogenic materials in the sample may be captured by the magnetic immunoparticles.

The magnetic field-forming unit 140 may include a material 141 capable of applying a magnetic field at one side. For example, in the magnetic field-forming unit 140, a magnet may be disposed at the outside as the material 141 capable of applying a magnetic field to the magnetic field-forming unit 140. Accordingly, as shown in FIG. 19, magnetic immunoparticles bound to pathogenic materials or magnetic immunoparticles not bound to pathogenic materials may be moved to one side of the magnetic field-forming unit 140 in which the magnet is disposed.

The first discharge unit (also referred to as a “sample discharge unit”) 150 is a unit through which a sample (e.g., blood) is discharged, and the sample discharged from the first discharge unit 150 may be injected back into the patient. The first discharge unit 150 is branched from the magnetic field-forming unit 140, and due to the magnetic immunoparticles bound to pathogenic materials and magnetic immunoparticles not bound to pathogenic materials which have moved to one side of the magnetic field-forming unit 140, the sample from which the magnetic immunoparticles and the pathogenic materials have been removed may be discharged from the first discharge unit 150.

From the second discharge unit 160, other sample than the sample discharged through the first discharge unit 150 may be discharged. In other words, the magnetic immunoparticles bound to pathogenic materials or magnetic immunoparticles not bound to pathogenic materials which have been moved to one side of the magnetic field-forming unit 140 may be discharged through the second discharge unit 160.

As described above, the fluidic device 100 according to a specific embodiment may capture pathogenic materials using the above-described magnetic immunoparticles, and may separate and discharge the captured pathogenic materials from the sample, thereby supplying the purified sample back into the patient. In addition, since the captured pathogenic materials are discharged through the second discharge unit 160, the fluidic device 100 may be used semi-permanently without the need to replace.

Referring to FIG. 20, a fluidic device 100A according to a specific embodiment may include a collecting unit 170, instead of the second discharge unit 160. The fluidic device 100A according to a specific embodiment is different from the fluidic device 100 of the above-described embodiment in that it does not include the second discharge unit 160 and includes a collecting unit 170. Other configuration of the fluidic device 100A may be the same as that of the fluidic device 100, and detailed descriptions thereof will be omitted.

As shown in FIG. 20, magnetic immunoparticles bound to pathogenic materials or magnetic immunoparticles not bound to pathogenic materials move to one side of the magnetic field-forming unit 140 by a material 141 capable of applying a magnetic field. The collecting unit 170 may be disposed on one side such that they are not discharged through the first discharge unit (also referred to as a “sample discharge unit”) 150. In other words, the collecting unit 170 may constitute an obstacle to prevent pathogenic materials from being discharged to the outside of the fluidic device 100A.

Through this configuration, the pathogenic materials are maintained in a state of being collected in the collecting unit 170, and it is possible to more reliably prevent them from entering the patient's body. The captured pathogenic materials may be removed during replacement of the fluidic device 100.

Although the collecting unit 170 is shown to have a flat surface as in FIG. 20, a surface inclined with respect to the sample inflow direction may be provided to more easily collect pathogenic materials. Alternatively, the collecting unit 170 may have irregularities which are formed to more easily collect pathogenic materials.

Still another aspect provides a machine for treating an infectious disease, the device including the magnetic immunoparticles. The machine for treating an infectious disease according to a specific embodiment may be implemented in the form of a magnetic immunoparticle-based extracorporeal circulation machine for removing pathogenic materials, including the fluidic device 100 (also referred to as a magnetic immunoparticle-based “hemodialysis machine”).

A schematic illustration of the magnetic immunoparticle-based extracorporeal circulation machine for removing pathogenic materials is as shown in FIG. 21 or 22, but is not limited thereto, and it may be modified, as long as the same principle is applied.

More specifically, referring to FIG. 21, the magnetic immunoparticle-based extracorporeal circulation machine for removing pathogenic materials (also referred to as a magnetic immunoparticle-based “hemodialysis machine”) 1000 may include the fluidic device 100, and for example, the fluidic device 100 may be provided with the above-described configuration, i.e., the injection unit, the reaction unit, the magnetic field-forming unit, the first discharge unit (also referred to as the “sample discharge unit”), and the second discharge unit. The magnetic immunoparticle-based extracorporeal circulation machine for removing pathogenic materials 1000 has a structure in which a sample (e.g., blood) and a dialysate may pass through the inside, and may include a hemodialysis filter 1200 that discharges impurities in the blood into the dialysate, a blood pump 1100 for pumping the patient's blood to the hemodialysis filter 1200, a dialysate supply tank 1500 for storing clean dialysate, a dialysate recovery tank 1400 for storing the dialysate passed through the hemodialysis filter 1200, and/or a dialysate pump 1300 for supplying the dialysate to the hemodialysis filter 1200 and recovering the dialysate from the hemodialysis filter 1200. The hemodialysis filter 1200, the blood pump 1100, and the dialysate pump 1300 are connected to each other by connectors, and in the connectors, a part at which blood flows into the hemodialysis filter 1200, a part at which blood flows out from the hemodialysis filter 1200, a part at which the dialysate flows into the hemodialysis filter 1200, and a part at which the dialysate flows out from the hemodialysis filter 1200 may be connected with a pressure gauge (not shown) for measuring the pressure of blood or dialysate, respectively.

Such a magnetic immunoparticle-based extracorporeal circulation machine for removing pathogenic materials (also referred to as a magnetic immunoparticle-based “hemodialysis machine”) 1000 may discharge impurities from the blood to the outside while substances move between the blood and dialysate inside the hemodialysis filter 1200. The fluidic device 100 for removing the pathogenic materials may be connected between any components in the magnetic immunoparticle-based extracorporeal circulation machine 1000 for removing pathogenic materials, e.g., between the blood pump 1100 and the hemodialysis filter 1200, or between the hemodialysis filter 1200 and the dialysate pump 1300. In addition, the magnetic immunoparticle-based extracorporeal circulation machine 1000 for removing pathogenic materials may include at least one or two or more of the fluidic device 100 for removing pathogenic materials.

The magnetic immunoparticle-based extracorporeal circulation machine 1000 for removing pathogenic materials, the machine including the fluidic device 100 may remove pathogenic materials in the blood by using the magnetic immunoparticles, and may prevent the magnetic immunoparticles from being injected into the body by using the magnetic field, and thus has the effect of injecting the clean blood, from which the pathogenic materials have been removed, back into an individual.

Specifically, referring to FIG. 22, in the magnetic immunoparticle-based extracorporeal circulation machine for removing pathogenic materials (also referred to as a magnetic immunoparticle-based “hemodialysis machine”) 1000′, the hemodialysis filter 1200 may be replaced by the fluidic device 100 for removing pathogenic materials, or no dialysate may be used while replacing the hemodialysis filter 1200 by the fluidic device 100 for removing pathogenic materials.

More specifically, the magnetic immunoparticle-based extracorporeal circulation machine for removing pathogenic materials 1000′ has a configuration in which the dialysate supply tank 1500 for storing dialysate, the hemodialysis filter 1200 discharging impurities in the blood into the dialysate, the dialysate recovery tank 1400 for storing the dialysate passed through the hemodialysis filter 1200, and the dialysate pump 1300 for transporting the dialysate to the hemodialysis filter 1200 are excluded, and has a configuration including the pump 1100 for pumping blood to the fluidic device 100 for removing pathogenic materials from the blood, an inlet through which blood is introduced, an outlet through which the blood is discharged, and the fluidic device 100 connecting the inlet and the outlet, inside which the blood flows and pathogenic materials are captured or removed.

Among the terms or elements mentioned in the composition for treatment, the treatment method, and the device for treatment, those the same as mentioned in the description of the magnetic immunoparticles, the composition, the method, or the kit are understood to be the same as mentioned in the above description of the magnetic immunoparticles, the composition, the method, or the kit.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 shows a schematic illustration of a method of producing magnetic immunoparticles according to one exemplary embodiment;

FIG. 2 shows transmission electron microscope (TEM) images of magnetic immunoparticles produced according to one exemplary embodiment, wherein the left image shows magnetic particles used in one exemplary embodiment, and the right image shows magnetic immunoparticles produced according to one exemplary embodiment;

FIG. 3 is a graph showing a bacteria removal rate (%) by using the magnetic immunoparticles according to one exemplary embodiment, wherein D-HL represents the use of cell membranes of differentiated HL60 cells, and C-HL represents the use of cell membranes of undifferentiated HL60 cells, E+V as a comparative group represents an experimental group treated with cell membranes to which magnetic particles had not been attached, and E+V+M represents an experimental group treated with magnetic immunoparticles to which magnetic particles had been attached;

FIG. 4 shows a schematic illustration of a method of collecting, concentrating, and removing pathogenic materials in blood, based on the magnetic immunoparticles according to one exemplary embodiment;

FIG. 5 is a graph showing a removal rate (%) of MRSA, which is Gram-positive bacteria, by using the magnetic immunoparticles according to one exemplary embodiment;

FIG. 6 is a graph showing a removal rate (%) of ESBL-EC, which is Gram-negative bacteria, by using the magnetic immunoparticles according to one exemplary embodiment;

FIG. 7 is a graph showing a removal rate (%) of HCoV229E virus by using the magnetic immunoparticles according to one exemplary embodiment;

FIG. 8 is a graph showing the ability of the magnetic immunoparticles according to one exemplary embodiment to remove pathogenic bacteria (MRSA) in diabetic blood;

FIG. 9 is a graph showing the ability of the magnetic immunoparticles according to one exemplary embodiment to remove a pathogenic virus (CMV) in diabetic blood;

FIG. 10 shows a schematic illustration of a method of removing pathogenic materials by magnetic immunoparticle-based extracorporeal circulation according to one exemplary embodiment;

FIG. 11 is a graph showing the ability to remove pathogenic bacteria (MRSA) in blood in vitro at the time of using the method of removing pathogenic materials by magnetic immunoparticle-based extracorporeal circulation according to one exemplary embodiment;

FIG. 12 is a graph showing the ability to remove a pathogenic virus (CMV) in blood in vitro when using the method of removing pathogenic materials by magnetic immunoparticle-based extracorporeal circulation according to one exemplary embodiment;

FIG. 13 is a graph showing the ability to remove a pathogenic material (LPS) in blood in vitro when using the method of removing pathogenic materials by magnetic immunoparticle-based extracorporeal circulation according to one exemplary embodiment;

FIG. 14 is a graph showing the ability to remove a pathogenic material (ZIKV protein) in blood in vitro when using the method of removing pathogenic materials by magnetic immunoparticle-based extracorporeal circulation according to one exemplary embodiment;

FIG. 15 is a graph showing the ability to remove a pathogenic material (SARS-CoV-2 Spike protein) in blood in vitro when using the method of removing pathogenic materials by magnetic immunoparticle-based extracorporeal circulation according to one exemplary embodiment;

FIG. 16 shows a schematic illustration of the application, to an animal, of the method of removing pathogenic materials by magnetic immunoparticle-based extracorporeal circulation according to one exemplary embodiment;

FIG. 17 is a graph showing the ability to remove pathogenic bacteria (MRSA) in blood of a rat animal in vivo when using the method of removing pathogenic materials by magnetic immunoparticle-based extracorporeal circulation according to one exemplary embodiment;

FIG. 18 shows a schematic illustration of a magnetic immunoparticle-based extracorporeal circulation method using a fluidic device 100, in which P represents a pump;

FIG. 19 shows a schematic illustration of an example of the fluidic device 100;

FIG. 20 shows a schematic illustration of another example of the fluidic device 100A;

FIG. 21 shows a schematic illustration of a magnetic immunoparticle-based extracorporeal circulation machine 1000 for removing pathogenic materials, the machine including the fluidic device 100; and

FIG. 22 shows a schematic illustration of a magnetic immunoparticle-based extracorporeal circulation machine 1000′ for removing pathogenic materials, the machine including the fluidic device 100.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Hereinafter, the present disclosure will be described in more detail with reference to exemplary embodiments. However, these exemplary embodiments are for illustrative purposes only, and the scope of the present disclosure is not intended to be limited by these exemplary embodiments. It is apparent to those skilled in the art that various changes may be made therein without departing from the spirit and scope of the present disclosure.

### EXAMPLES

### Example 1: Preparation of Magnetic Immunoparticles

Magnetic immunoparticles were prepared using human red blood cells in the blood of a human living body as a model. Red blood cells were obtained from the Red Cross (South Korea). Red blood cells were suspended in 1 mL of a 25% v/v mixture of PBS (pH 7.2, Biosesang, South Korea) and distilled water (Biosesang, South Korea) at a density of 10⁸ cells, and treated with a low osmotic pressure at 4° C. for 1 hour, and then centrifuged at 4° C. for 5 minutes (Centrifuge 5424R, Eppendorf, Germany) and prepared in 1×PBS.

In addition, cell membranes separated (purified) by the low osmotic pressure treatment were subjected to sonication (0700 Ultra-Sonicator, Qsonica, USA) for 10 minutes at 4° C. 20 kHz. and 150 W to split the cell membranes into smaller pieces.

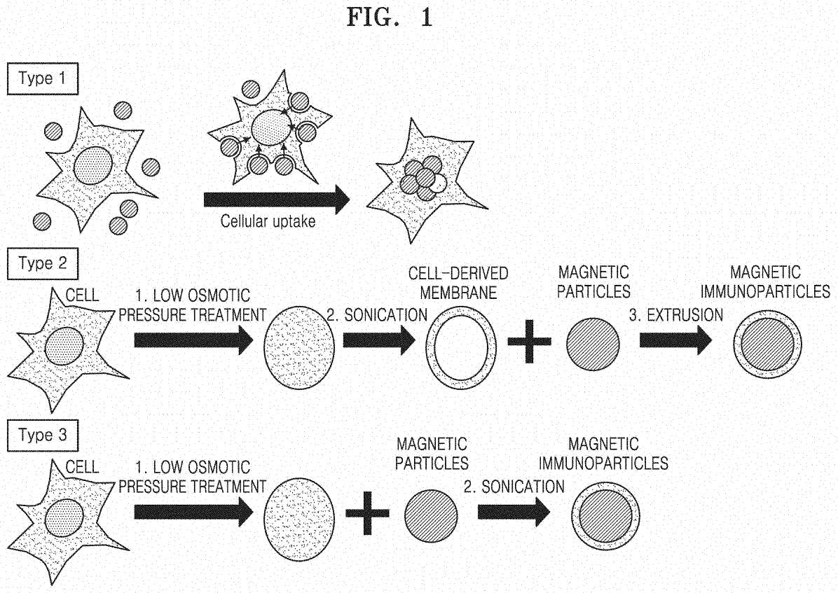

Further, the prepared red blood cell-derived cell membranes were extruded together with magnetic particles in an Avanti mini extruder (Avanti Polar Lipids, Alabaster, Ala., USA) using 1 μm, 0.4 μm, and 0.2 μm pore size track-etched membrane filters sequentially to prepare magnetic immunoparticles. In detail, according to Type 2 as shown in FIG. 1, the prepared cell membranes and magnetic particles (0.5 mg/mL) were mixed and extruded to prepare magnetic immunoparticles in which the magnetic particles were invaginated into the cell membranes. As the magnetic particles, iron oxide magnetic particles (Carboxyl-Adembeads 200 nm, Ademtech, France) having an average particle size of 200 nm, of which surface was modified with carboxylic acid, were used.

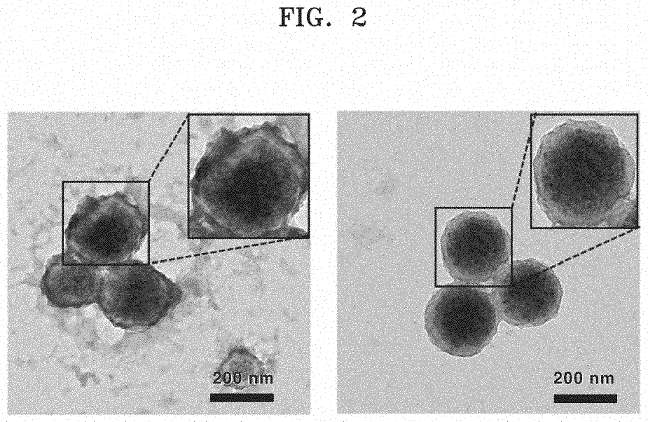

As a result, as shown in FIG. 2, transmission electron microscope (TEM) images showed that magnetic immunoparticles (right image of FIG. 2) having an average size of about 250 nm were generated, in which the magnetic particles were invaginated into the cell-derived membranes.

### Example 2: Test of Microbial Removal Ability of Magnetic Immunoparticles

E. coli (1×10⁸ CFU/mL) was inoculated in a solution containing the magnetic immunoparticles prepared in Example 1 and reacted for 2 hours at 15 rpm in an incubator at a temperature of 37° C.

Each reacted solution was transferred to a 1 mL tube (Eppendorf), and then a permanent magnet was attached to one side of the outer surfaces of the tube to apply a magnetic field to the tube for 40 minutes. After applying the magnetic field for 40 minutes, the solution was extracted from the tube on the opposite side of the permanent magnet attached to the tube, and each 100 μl of the extracted solution was plated on an LB agar plate, and the number of bacterial colonies was observed and quantified after overnight incubation. For comparison, magnetic immunoparticles prepared using undifferentiated HL60 cells or differentiated HL60 cells prepared in Example 1, differentiated HL60 cells or undifferentiated HL60 cells were used to allow the reaction of Example 2, respectively. A reduction rate (%) was expressed as a percentage of (1-(the number of E. coli after treatment with magnetic immunoparticles/the number of E. coli before treatment with magnetic immunoparticles)).

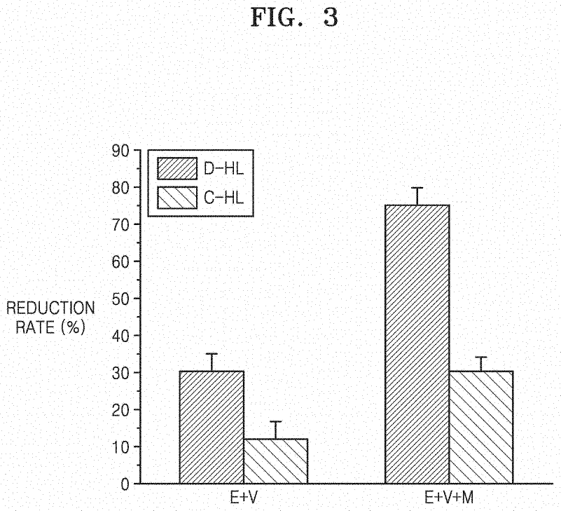

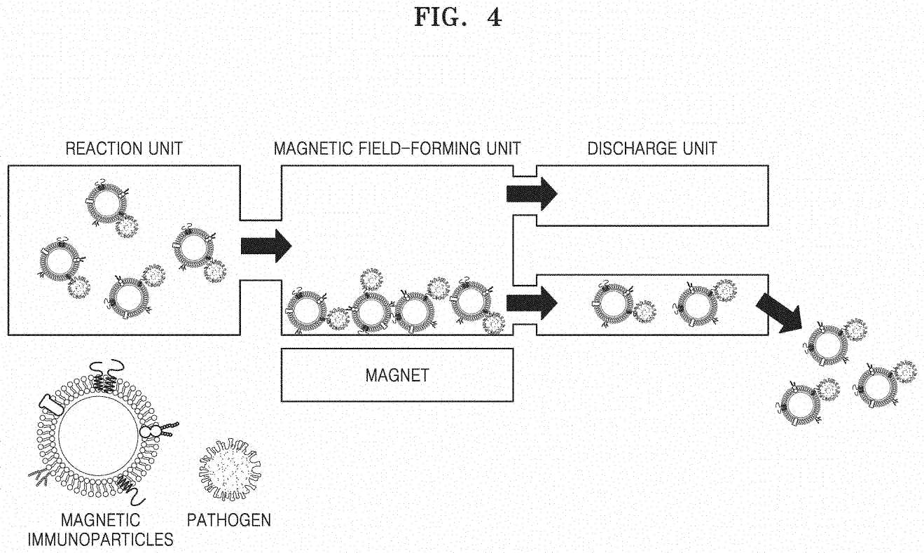

As a result, as shown in FIG. 3, it was confirmed that when the magnetic immunoparticles prepared by using the cell membrane of differentiated HL60 cells were used, the E. coli removal rate was about 75%, indicating an excellent microbial removal efficiency.

### Example 3-Example 12: Preparation of Magnetic Immunoparticles Using Various Cells

In the present exemplary embodiments, various kinds of magnetic immunoparticles were prepared using the following cells according to the method of Example 1:

human red blood cell (RBC); human U937-differentiated M0 macrophage: M0 macrophage-like cell obtained by differentiating human U937 cell line (leukemia cell line): human U937-differentiated M1 macrophage: M1 macrophage-like cell obtained by differentiating human U937 cell line; human U937-differentiated M2 macrophage: M2 macrophage-like cell obtained by differentiating human U937 cell line; human THP-1-differentiated M0 macrophage: M0 macrophage-like cell obtained by differentiating human THP-1 cell line (human monocyte cell line derived from a patient with acute monocytic leukemia); human HL-60-differentiated neutrophil: neutrophil-like cell obtained by differentiating human HL-60 cell line (leukemia cell line); human K562 cell line (leukemia cell line); human oral epithelial cell; human hepatic sinusoidal endothelial cell (HSEC); or human intestinal epithelial cell line (Caco-2).

As a result, as shown in Table 1 below, a total of 10 types of magnetic immunoparticles were obtained using cell membranes derived from the above various cells.

TABLE 1

| Magnetic | |
|---|---|
| immunoparticles | Cell membrane |
| Example 3 | Cell membrane of human RBC |
| Example 4 | Cell membrane of human |
| | U937-differentiated M0 |
| | macrophage |
| Example 5 | Cell membrane of human |
| | U937-differentiated M1 |
| | macrophage |
| Example 6 | Cell membrane of human |
| | U937-differentiated M2 |
| | macrophage |
| Example 7 | Cell membrane of human |
| | THP-1-differentiated M0 |
| | macrophage |
| Example 8 | Cell membrane of human |
| | HL-60-differentiated |
| | neutrophil |
| Example 9 | Cell membrane of human K562 |
| Example 10 | Cell membrane of human |
| | hepatic sinusoidal endothelial |
| | cell (HSEC) |
| Example 11 | Cell membrane of human |
| | intestinal epithelial cell (Caco-2) |
| Example 12 | Cell membrane of human |
| | oral epithelial cell |

### EXPERIMENTAL EXAMPLES

### Experimental Example 1: Test of Removal Ability of Magnetic Immunoparticles Against Gram-Positive/Negative Bacteria

In this Experimental Example, to examine whether the magnetic immunoparticles of Table 1 are able to remove pathogens in the blood, each of the magnetic immunoparticles of Table 1 was independently injected into a human blood sample containing bacteria and the bacteria captured by the magnetic immunoparticles were removed by applying a magnetic field, and then changes of colony forming unit (CFU) of the inoculated bacteria in the sample were measured.

In detail, methicillin resistant Staphylococcus aureus (MRSA) which is a Gram-positive bacterium or extended-spectrum beta-lactamase-producing Escherichia coli (ESBL-EC) which is a Gram-negative bacterium was inoculated in 1 mL of an anticoagulant-treated human blood (Red Cross, South Korea) sample at a concentration of 10⁴ CFU/mL, and incubated at 37° C. for 10 minutes. Each of the magnetic immunoparticles of Table 1 was independently injected into the incubated blood sample such that the final concentration of the magnetic immunoparticles was 150 μg/mL. The equivalent amount of physiological saline was injected into a control group. Thereafter, after a reaction for 20 minutes at 37° C., the magnetic immunoparticles in the blood sample were fixed at a specific position using a magnet for 15 minutes to prevent the magnetic immunoparticles from being included in the supernatant, and then the supernatant was collected. CFU of bacteria in the supernatant was examined. In detail, the supernatant (100 μL) of the blood sample was diluted with 900 μL of physiological saline, and plated on LB agar medium using a microbial analyzer (EDDY JET2, IUL micro, USA), and incubated at 37° C. for 24 hours. Thereafter, CFU of the bacteria on the LB agar medium was measured using a microbial colony counter (Sphereflash colony counter and zone reader, IUL micro, USA).

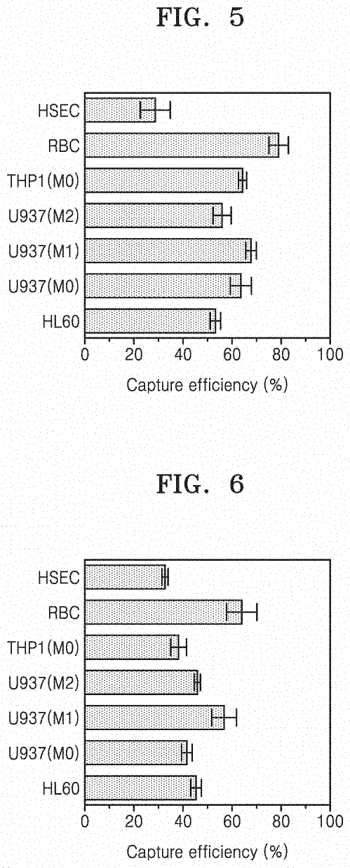

As a result, as shown in FIGS. 5 and 6, it was confirmed that most of the magnetic immunoparticles are able to remove MRSA and ESBL-EC in the blood sample by capturing. In particular, it was confirmed that magnetic immunoparticles (Example 3) prepared using cell membranes of human red blood cells (RBC) showed the most excellent removal ability against MRSA and ESBL-EC in blood samples. In detail, it was confirmed that the magnetic immunoparticles (Example 3) prepared using the cell membrane of human red blood cells (RBC) are able to remove about 75% or more of MRSA Gram-positive bacterium or about 85% or more of Gram-negative bacteria ESBL-EC in the blood sample.

### Experimental Example 2: Test of Removal Ability of Magnetic Immunoparticles Against Viruses

In this Experimental Example, to examine whether the magnetic immunoparticles of Table 1 are able to remove viruses in the blood, each of the magnetic immunoparticles of Table 1 was independently injected into a human blood sample containing viruses, and the viruses captured by the magnetic immunoparticles were removed by applying a magnetic field, and then changes in the amounts of RNA of the inoculated viruses in the culture medium were measured.

In detail, HCoV229E (Human Coronavirus 229E) was inoculated in 1 mL of an anticoagulant-treated human blood (Red Cross, South Korea) sample at a density of 10′ PFU/mL, and incubated at 37° C. for 10 minutes. Each of the magnetic immunoparticles of Table 1 was independently injected into the incubated blood sample such that the final concentration of the magnetic immunoparticles was 150 μg/mL. The equivalent amount of physiological saline was injected into a control group. Thereafter, after a reaction for 20 minutes at 37° C., the magnetic immunoparticles in the blood sample were fixed at a specific position using a magnet for 15 minutes to prevent the magnetic immunoparticles from being included in the supernatant, and then the supernatant was collected. The amount of RNA of viruses in the supernatant was examined. Nucleic acids were extracted from viruses in the supernatant using a QIAmp viral RNA mini kit (QIAGEN, Germany), and the extracted nucleic acids were amplified using SYBR PCR master mix (Toyobo, Japan) and Real time PCR (CFX connect, BIO-RAD, USA) to measure the amount of RNA.

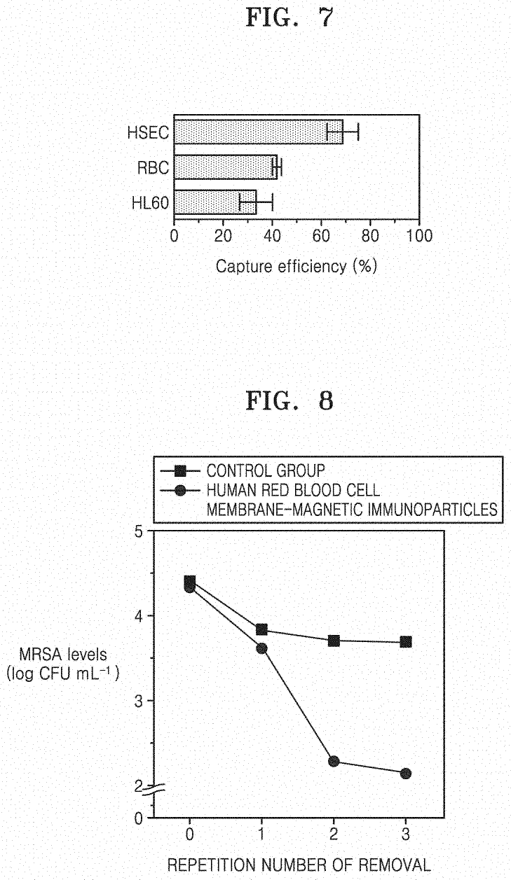

As a result, as shown in FIG. 7, it was confirmed that the magnetic immunoparticles (Example 3) prepared using the cell membrane of human red blood cells (RBC), the magnetic immunoparticles (Example 10) prepared using the cell membrane of human hepatic sinusoidal endothelial cells (HSEC), and the magnetic immunoparticles (Example 8) prepared using the cell membrane of human HL-60-differentiated neutrophil are able to remove HCoV229E virus in the blood sample by capturing. In particular, it was confirmed that magnetic immunoparticles (Example 10) prepared using cell membranes of human hepatic sinusoidal endothelial cells (HSEC) showed the most excellent removal ability against HCoV229E virus in the blood sample. In detail, it was confirmed that magnetic immunoparticles (Example 10) prepared using the cell membrane of human hepatic sinusoidal endothelial cells (HSEC) are able to remove about 70% or more of HCoV229E virus in the blood sample by capturing.

### Experimental Example 3: Test of Removal Ability of Magnetic Immunoparticles Against Pathogens (Bacteria or Viruses) in Diabetic Blood

In this Experimental Example, to examine whether the magnetic immunoparticles of Table 1 are able to remove pathogens (bacteria or viruses) in the diabetic blood, pathogens were arbitrarily inoculated into a human blood sample to which glucose (D-glucose, Sigma-Aldrich, USA) was arbitrarily added, and then cultured. Each of the magnetic immunoparticles of Table 1 were independently injected to the culture medium, and the pathogens captured by the magnetic immunoparticles were removed by applying a magnetic field. Thereafter, changes in the concentration of the pathogens in the culture medium were measured.

In detail, D-glucose was added in 1 mL of an anticoagulant-treated human blood (Red Cross, South Korea) sample at a concentration of about 400 mg/dL to about 450 mg/dL, and incubated at 37° C. for 10 minutes. Pathogens (bacteria or viruses) were inoculated in the incubated blood sample at a concentration of 10⁴ CFU/mL (or 105 PFU/mL), and incubated at 37° C. for 10 minutes. Each of the magnetic immunoparticles of Table 1 was independently injected into the incubated blood sample such that the final concentration of the magnetic immunoparticles was 150 μg/mL. The equivalent amount of physiological saline was injected into a control group. Thereafter, after a reaction for 20 minutes at 37° C., the magnetic immunoparticles in the blood sample were fixed at a specific position using a magnet for 15 minutes to prevent the magnetic immunoparticles from being included in the supernatant, and then the supernatant was collected. The concentration of the pathogens in the supernatant was examined. Changes in the concentration of bacteria, among the pathogens, in the blood sample were determined by measuring CFU of the bacteria in the same manner as in Experimental Example 1, and changes in the concentration of viruses, among the pathogens, in the blood sample were determined by measuring the amount of RNA of the viruses in the same manner as in Experimental Example 2. As the pathogens inoculated in this Experimental Example, MRSA or Cytomegalovirus (CMV) was used, and as the injected magnetic immunoparticles, the magnetic immunoparticles (Example 3) prepared using the cell membrane of human red blood cell (RBC), the magnetic immunoparticles (Example 12) prepared using the cell membrane of human oral epithelial cell, and the magnetic immunoparticles (Example 10) prepared using the cell membrane of human hepatic sinusoidal endothelial cell (HSEC) were used.

As a result, as shown in FIG. 8, it was confirmed that the concentration of the MRSA pathogen in the diabetic blood sample was remarkably reduced by injecting the magnetic immunoparticles (Example 3) prepared using the cell membrane of human red blood cell (RBC), as compared with the control group, and the concentration of the MRSA pathogen was further reduced as the removal process was repeated.

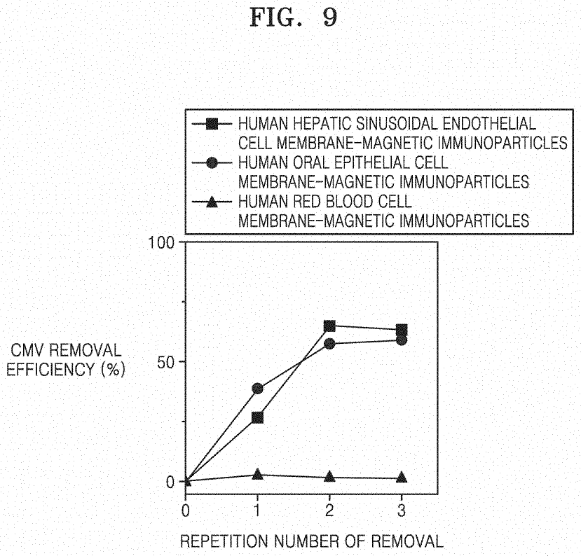

As shown in FIG. 9, it was also confirmed that the concentration of the CMV pathogen in the diabetic blood sample was remarkably reduced by injecting the magnetic immunoparticles (Example 12) prepared using the cell membrane of human oral epithelial cell and the magnetic immunoparticles (Example 10) prepared using the cell membrane of human hepatic sinusoidal endothelial cell (HSEC), and the concentration of the CMV pathogen was further reduced as the removal process was repeated.

This Experimental Example confirmed that the magnetic immunoparticles of Table 1 are able to effectively remove pathogens (bacteria or viruses) in the diabetic blood.

### Experimental Example 4: In Vitro Removal of Pathogens or Pathogenic Materials in Blood by Method of Removing Pathogenic Materials Using Magnetic Immunoparticle-Based Extracorporeal Circulation

In this Experimental Example, for in vitro removal of pathogens or pathogenic materials in a large amount of blood using the magnetic immunoparticles of Table 1, a method of removing pathogenic material using magnetic immunoparticle-based extracorporeal circulation was used.

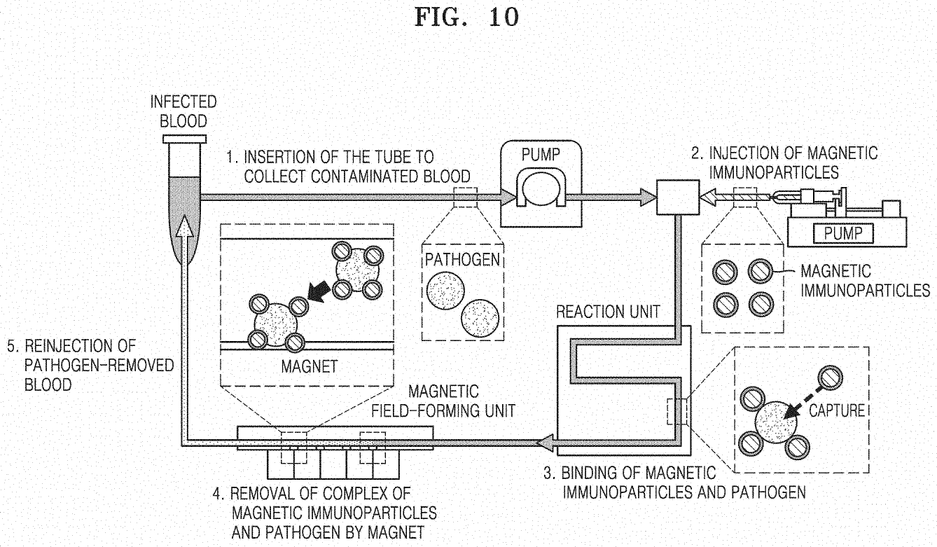

The method of removing pathogenic material using magnetic immunoparticle-based extracorporeal circulation includes, as shown in FIG. 10, binding the pathogens or pathogenic materials with the magnetic immunoparticles during a process of mixing the pathogens or pathogenic material-contaminated blood with the magnetic immunoparticles in a reaction unit (a fluid element for mixing) of the magnetic immunoparticle-based extracorporeal circulation machine for removing pathogenic materials; and removing the complex bound to the pathogens or pathogenic materials to the magnetic immunoparticles from the blood by a magnet during a process of passing the blood including the complex through a magnetic field-forming unit (a fluid element for magnetophoretic separation). Blood may be purified by repeating the process in which each procedure is sequentially performed in vitro.

In detail, bacteria (10⁴ CFU/mL), viruses (PFU/mL), or inflammatory materials (LPS, 10 μg/mL) were inoculated in 10 mL of anticoagulant-treated human blood (Red Cross, South Korea) or whole blood of a rat (8-week-old, male), and incubated at 37° C. for 10 minutes. In addition, a solution containing the magnetic immunoparticles of Table 1 was prepared in saline at a concentration of 0.5 mg/mL. The incubated blood sample and the prepared magnetic immunoparticle solution were injected into a magnetic immunoparticle-based extracorporeal circulation machine for removing pathogenic materials, and loaded at a rate of 10 mL/hr and 0.5 mL/hr, respectively. When the injected blood sample and the magnetic immunoparticle solution were mixed while running through the reaction unit (the fluid element for mixing), the pathogens or pathogenic materials in the blood sample were bound to the magnetic immunoparticles. The complexes bound to the pathogens or pathogenic materials to the magnetic immunoparticles in the blood were captured toward the magnet by a magnetic field while passing through the magnetic field-forming unit (fluid element for magnetophoretic separation), such that the complexes were removed from the blood sample. The blood sample from which the pathogens or pathogenic materials had been removed was collected, and then injected again into the magnetic immunoparticle-based extracorporeal circulation machine for removing pathogenic materials, and the process of removing pathogenic materials by the above magnetic immunoparticle-based extracorporeal circulation was repeated for 5 hours. At the same time, the concentrations of the pathogens or pathogenic materials in the blood sample were measured every hour. The change in the concentrations of bacteria, among pathogens, in the blood sample was determined by measuring CFU of the bacteria in the same manner as in Experimental Example 1, and the change in the concentrations of viruses, among pathogens, in the blood sample was determined by measuring the amount of RNA of the viruses in the same manner as in Experimental Example 2. In addition, changes in the concentrations of LPS, ZIKV Protein, or SARS-CoV-2 Spike Protein as pathogenic materials in the blood samples were determined by enzyme-linked immunosorbent assay (ELISA), and an LPS ELISA kit (LS-F55757-1, LSbio, USA), a Zika virus (strain Zika SPH2015) Envelope Protein (ZIKV-E) ELISA Kit (Sinobio, China), or a SARS-CoV-2 Spike protein ELISA kit (ab274342, abcam, USA) was used.

In this Experimental Example, when MRSA as a bacterial pathogen was inoculated in human blood or rat blood samples, in order to remove the MRSA, the magnetic immunoparticles (Example 3) prepared using the cell membrane of human red blood cells (RBC) or the magnetic immunoparticles prepared using the cell membrane of red blood cells of a Wistar rat (8 weeks old, male, Orient Bio, South Korea) were used as the magnetic immunoparticles.

In this Experimental Example, when CMV as a viral pathogen was inoculated in the blood sample, in order to remove the CMV, the magnetic immunoparticles (Example 10) prepared using the cell membrane of human hepatic sinusoidal endothelial cells (HSEC) were used as magnetic immunoparticles.

In this Experimental Example, when LPS as a pathogenic material was inoculated in the blood sample, in order to remove the LPS, the magnetic immunoparticles (Example 3) prepared using the cell membrane of human red blood cells (RBC) were used as magnetic immunoparticles.

In this Experimental Example, when ZIKV protein as a pathogenic material was inoculated in the blood sample, in order to remove the ZIKV protein, the magnetic immunoparticles (Example 10) prepared using the cell membrane of human hepatic sinusoidal endothelial cells (HSEC) or the magnetic immunoparticles (Example 9) prepared using the cell membrane of human K562 were used as magnetic immunoparticles.

In this Experimental Example, when SARS-CoV-2 Spike protein as a pathogenic material was inoculated in the blood sample, in order to remove the SARS-CoV-2 Spike protein, the magnetic immunoparticles (Example 10) prepared using the cell membrane of human hepatic sinusoidal endothelial cells (HSEC), the magnetic immunoparticles (Example 11) prepared using the cell membrane of human intestinal epithelial cells (Caco-2), or the magnetic immunoparticles (Example 7) prepared using the cell membrane of human THP-1-differentiated M0 macrophage were used as magnetic immunoparticles.

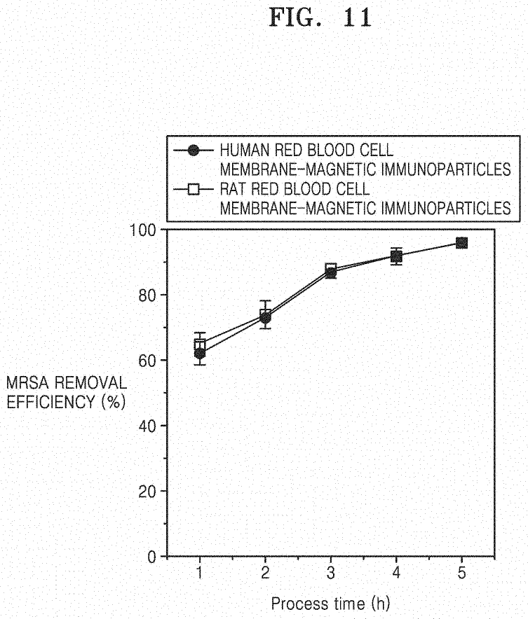

As a result, as shown in FIG. 11, it was confirmed that the magnetic immunoparticles (Example 3) prepared using the cell membrane of human red blood cells (RBC) or the magnetic immunoparticles prepared using the cell membrane of red blood cells of a rat are able to remove about 90% or more of MRSA in the human blood or rat blood.

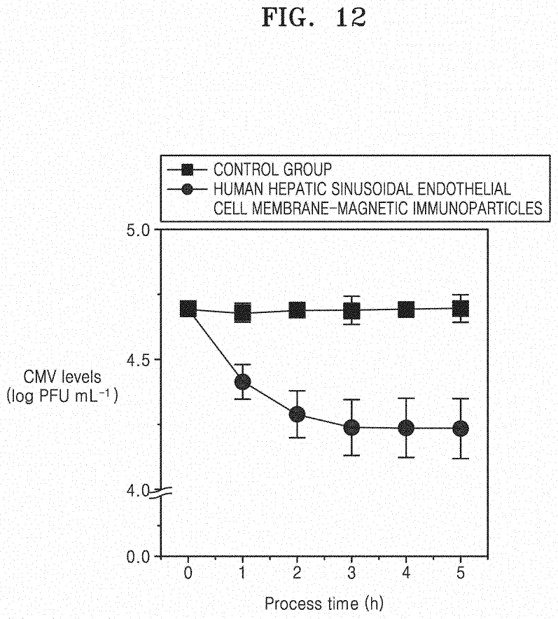

As shown in FIG. 12, it was also confirmed that the magnetic immunoparticles (Example 10) prepared using the cell membrane of human hepatic sinusoidal endothelial cells (HSEC) are able to remove about 66% or more of CMV in the blood.

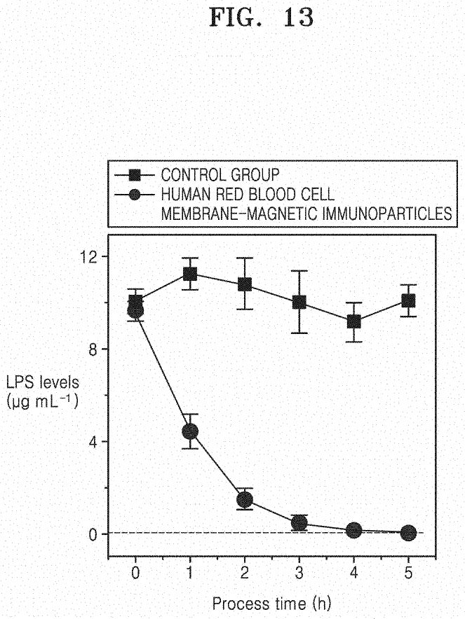

As shown in FIG. 13, it was also confirmed that the magnetic immunoparticles (Example 3) prepared using the cell membrane of human red blood cells (RBC) are able to remove about 90% or more of LPS in the blood.

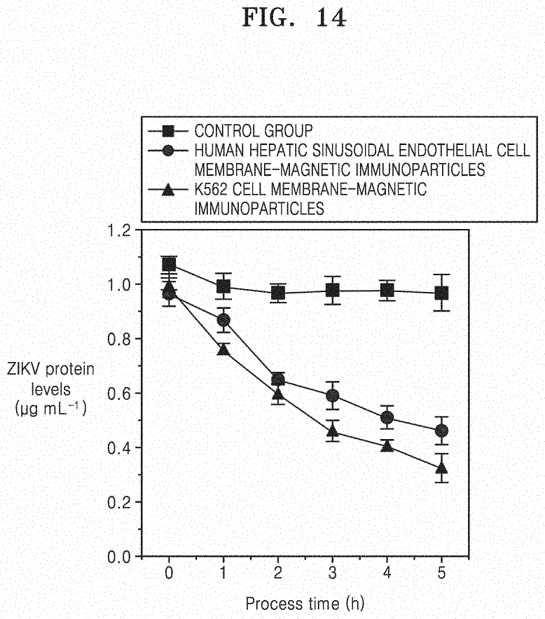

As shown in FIG. 14, it was also confirmed that the magnetic immunoparticles (Example 10) prepared using the cell membrane of human hepatic sinusoidal endothelial cells (HSEC) or the magnetic immunoparticles (Example 9) prepared using the cell membrane of human K562 are able to remove about 50% or more of ZIKV protein in the blood.

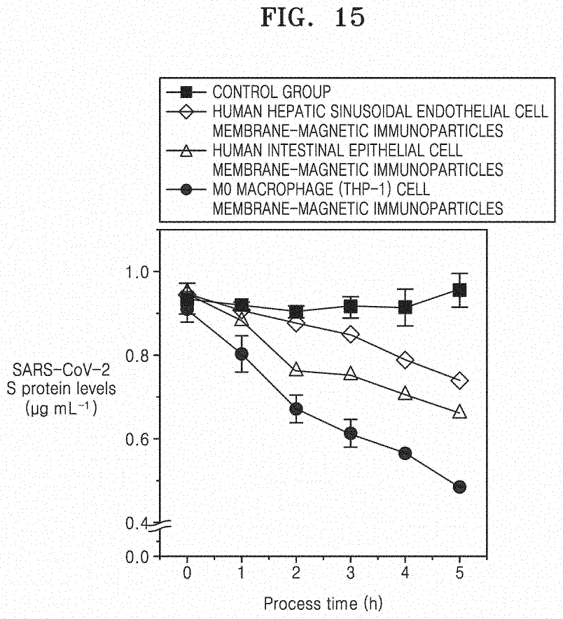

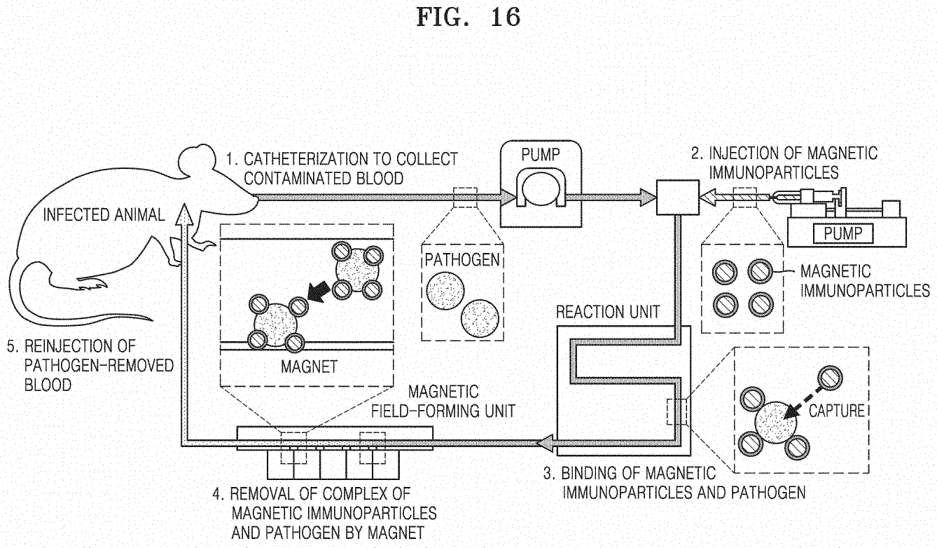

As shown in FIG. 15, it was also confirmed that the magnetic immunoparticles (Example 10) prepared using the cell membrane of human hepatic sinusoidal endothelial cells (HSEC) are able to remove about 20% or more of SARS-CoV-2 Spike protein in the blood, the magnetic immunoparticles (Example 11) prepared using the cell membrane of human intestinal epithelial cells (Caco-2) are able to remove about 30% or more of SARS-CoV-2 Spike protein in the blood, and the magnetic immunoparticles (Example 7) prepared using the cell membrane of human THP-1-differentiated M0 macrophage are able to remove about 50% or more of SARS-CoV-2 Spike protein in the blood.

This Experimental Example confirmed that pathogens (bacteria or viruses) or pathogenic materials in a large amount of blood may be effectively removed in vitro using the method of removing pathogenic materials by extracorporeal circulation based on the magnetic immunoparticles of Table 1.

### Experimental Example 5: In Vivo Removal of Pathogens or Pathogenic Materials in Blood by Method of Removing Pathogenic Materials Using Magnetic Immunoparticle-Based Extracorporeal Circulation

In this Experimental Example, for in vivo removal of pathogens or pathogenic materials in the blood using the magnetic immunoparticles of Table 1, a method of removing pathogenic material using magnetic immunoparticle-based extracorporeal circulation was used.

The method of removing pathogenic materials using magnetic immunoparticle-based extracorporeal circulation is the same as in Experimental Example 4, but is different from Experimental Example 4 in that the magnetic immunoparticle-based extracorporeal circulation machine for removing pathogenic materials was directly applied to a rat animal model arbitrarily infected with bacteria, and an in vivo test was performed in this Experimental Example.

In detail, as shown in FIG. 18, a catheter was inserted into the jugular vein of a normal Wistar rat (10-week-old, male) through surgery, and MRSA was arbitrarily injected through the catheter to infect the rat animal. In addition, a magnetic immunoparticle solution containing the magnetic immunoparticles prepared using the cell membrane of red blood cells derived from a Wistar rat (8-week-old, male) at a concentration of 0.5 mg/mL was prepared in saline. The infected rat and the magnetic immunoparticle-based extracorporeal circulation machine for removing pathogenic materials were connected through the catheter. By injecting the prepared magnetic immunoparticle solution into the extracorporeal circulation machine for removing pathogenic materials, which was connected to the rat, the method of removing pathogenic materials using magnetic immunoparticle-based extracorporeal circulation was performed, and the blood from which the pathogenic materials had been removed was injected back into the rat connected with the extracorporeal circulation machine for removing pathogenic materials.

The whole blood was collected from the rat at regular intervals (0 minutes, 15 minutes, 30 minutes, and 60 minutes) to measure changes in the concentrations of MRSA in the blood. The changes in the concentrations of MRSA was determined by measuring CFU of the bacteria in the whole blood sample collected in the same manner as in Experimental Example 1.

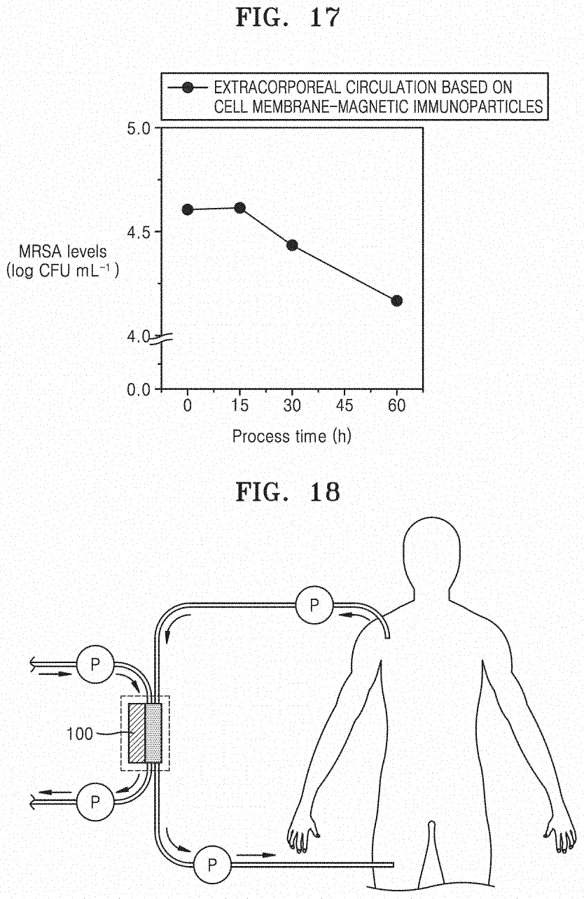

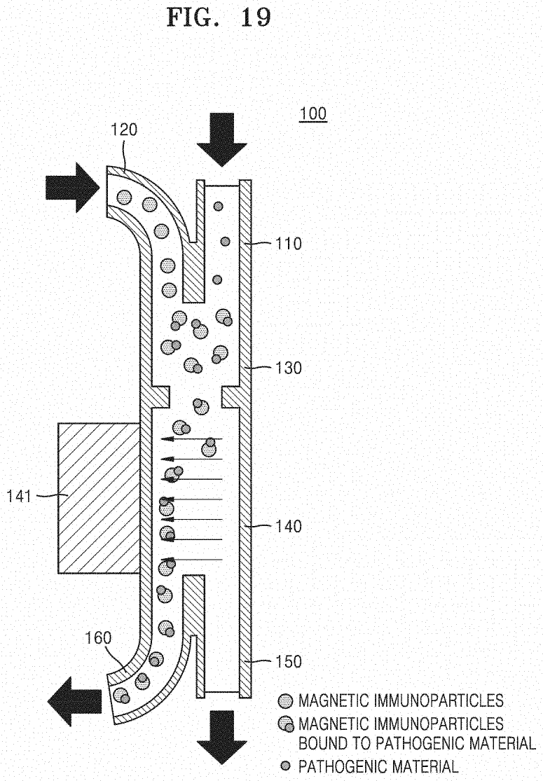

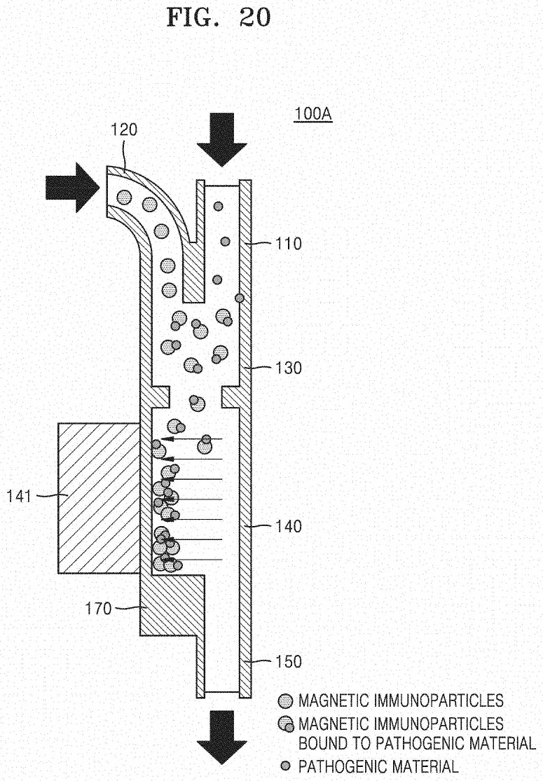

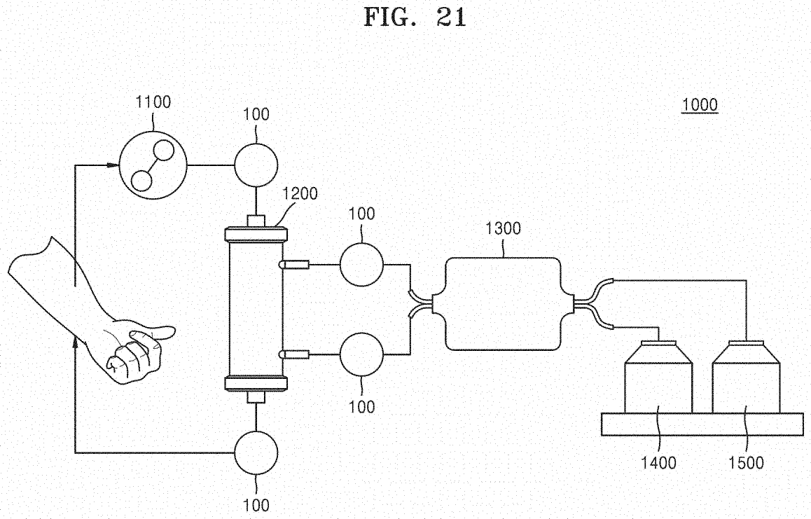

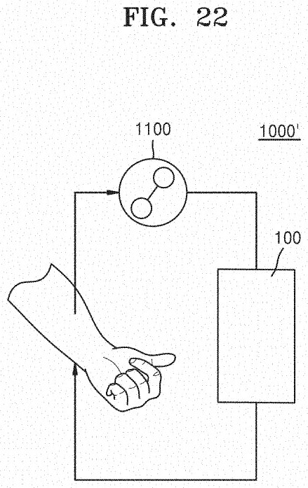

As a result, as shown in FIG. 17, it was confirmed that MRSA in the whole blood of the rat may be removed by directly applying the method of removing pathogenic materials using the magnetic immunoparticle-based extracorporeal circulation to the rat infected with MRSA.

This Experimental Example confirmed that pathogens (bacteria or viruses) or pathogenic materials in the blood may be effectively removed in vivo using the method of removing pathogenic materials by extracorporeal circulation based on the magnetic immunoparticles of Table 1, and as a result, infectious diseases may be treated.

Magnetic immunoparticles according to an aspect may include cell membranes derived from cells, and thus may minimize side effects in vivo, and may detect various kinds of pathogenic materials due to characteristics of the cells from which the cell membranes are derived. Further, since the magnetic immunoparticles include magnetic particles, the magnetic immunoparticles may be easily separated by applying a magnetic field, and thus pathogenic materials may be more effectively detected and removed. Furthermore, when the magnetic immunoparticles are used for treatment, the possibility of injection of the magnetic immunoparticles Into the body may be minimized, and thus side effects in vivo may be remarkably reduced.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. Magnetic immunoparticles comprising:
a cell membrane capable of capturing a pathogenic material; and
magnetic particles attached to the cell membrane,
wherein the cell membrane is derived from one or more selected from the group consisting of immune cells, red blood cells, endothelial cells, and epithelial cells.

2. The magnetic immunoparticles of claim 1, wherein the pathogenic material is one or more selected from the group consisting of pathogenic bacteria, fungi, viruses, parasites, prions, and toxins.

3. The magnetic immunoparticles of claim 1, wherein the immune cells are one or more selected from the group consisting of neutrophils, eosinophils, basophils, monocytes, lymphocytes, Kupffer cells, microglias, macrophages, dendritic cells, mast cells, B cells, T cells, natural killer cells (NK cells), immune cell-derived cell lines, immune cell-like cells, and stem cell-derived immune cells.

4. The magnetic immunoparticles of claim 1, wherein the magnetic particles comprise one or more magnetic elements selected from the group consisting of iron (Fe), nickel (Ni), cobalt (Co), manganese (Mn), bismuth (Bi), zinc (Zn), strontium (Sr), lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), ruthenium (Lu), copper (Cu), silver (Ag), gold (Au), cadmium (Cd), mercury (Hg), aluminum (Al), gallium (Ga), indium (in), thallium (TI), calcium (Ca), barium (Ba), radium (Ra), platinum (Pt), and lead (Pd).

5. The magnetic immunoparticles of claim 4, wherein the magnetic elements are oxidized or surface-modified with metals, functional groups, proteins, carbohydrates, polymers, or lipids.

6. The magnetic immunoparticles of claim 1, wherein the magnetic particles are comprised in a solution.

7. The magnetic immunoparticles of claim 1, comprising an outer surface comprising the cell membrane and an inner core comprising the magnetic particles.

8. The magnetic immunoparticles of claim 7, wherein the inner core comprises one or more magnetic particles.

9. The magnetic immunoparticles of claim 1, wherein the cell membrane forms a vesicle.

10. The magnetic immunoparticles of claim 1, wherein the cell membrane expresses one or more selected from the group consisting of lectins, Toll like receptors (TLRs), pattern recognition receptors (PRRs), cluster of differentiation (CD) molecules, neutrophil extracellular traps (NETs), glycophorins, and cytokine receptors.

11. The magnetic immunoparticles of claim 1, wherein the magnetic immunoparticles are used to detect or remove pathogenic materials.

12. A method of diagnosing an infectious disease, the method comprising bringing the magnetic immunoparticles of claim 1 into contact with a sample and mixing the magnetic immunoparticles with the sample, and applying a magnetic field to the mixed sample.

13. The method of claim 12, further comprising detecting pathogenic materials bound to the magnetic immunoparticles, wherein the pathogenic materials are one or more selected from the group consisting of pathogenic bacteria, fungi, viruses, parasites, prions, and toxins.

14. The method of claim 12, wherein the infectious disease is one or more selected from the group consisting of systemic or local infections, inflammation, sepsis, and poisoning by toxins.

15. A method of treating an infectious disease, the method comprising bringing the magnetic immunoparticles of claim 1 into contact with a sample and mixing the magnetic immunoparticles with the sample, and removing a pathogenic material by applying a magnetic field to the mixed sample.

16. The method of claim 15, wherein the pathogenic material is one or more selected from the group consisting of pathogenic bacteria, fungi, viruses, parasites, prions, and toxins.

17. The method of claim 15, wherein the infectious disease is one or more selected from the group consisting of systemic or local infection, Inflammation, sepsis, and poisoning by toxins.

18. The method of claim 15, wherein hemodialysis or extracorporeal circulation is applied to the method of treating an infectious disease.
